(19) United States
(12) Patent Application Publication
(10) Pub. No.: US 2024/0245487 A1
(43) Pub. Date: Jul. 25, 2024

(54) MEANS OF ATTACHING INSTRUMENTS TO A SURGICAL TABLE

(71) Applicant: Kyra Medical, Inc., Northborough, MA (US)

(72) Inventors: Jesse DRAKE, Westborough, MA (US); Ryan CUNNIFF, Hopkinton, MA (US); Jeremy Macpherson, Medfield, MA (US); Justin MCCARTHY, Boxborough, MA (US)

(73) Assignee: Kyra Medical, Inc., Northborough, MA (US)

(21) Appl. No.: 18/424,634

(22) Filed: Jan. 26, 2024

Related U.S. Application Data
(63) Continuation-in-part of application No. 17/683,884, filed on Mar. 1, 2022.
(60) Provisional application No. 63/207,489, filed on Mar. 3, 2021.

(51) Int. Cl. A61B 90/57

(52) U.S. Cl. CPC A61B 90/57; A61B 2090/571

(57) ABSTRACT

A mount configured to attach to a mount lug, the mount comprising accessory mount clamp comprising mount arm extending from the accessory mount clamp; and a fastener to connect the accessory mount clamp to the mount lug.

### RELATED APPLICATION

The present application claims priority under 35 U.S.C. § 120 to U.S. application Ser. No. 17/683,884 filed on Mar. 1, 2022, which claims priority under 35 U.S.C. § 119(e) to U.S. Provisional Application No. 63/207,489 filed on Mar. 3, 2021, which are herein incorporated by reference in their entireties.

### FIELD

The present disclosure relates generally to drainage systems for use in medical procedures and particularly for use in medical procedures in which lithotomy stirrups are employed.

### BACKGROUND

When performing a surgical procedure, and more particularly a urological surgical or a gynecological surgical procedure, clinicians may use fluids such as sterile saline for irrigation of the operative site. It is necessary to collect such fluids and record the amount of the fluid used during the procedure to ensure patient safety. Allowing such fluids to puddle or collect on the floor could present a hazard to the clinician and surgical team members. Many times, such procedures are performed with the patient in the supine position and with the patient's legs positioned up and away from the operative site near the perineum. In order to obtain access to the operative site the patient's legs may be positioned in lithotomy stirrups to optimize access to the operative site. Such stirrups are attached to the surgical table by clamps which are mounted at the distal end of the surgical table side rails.

Drainage bags, more particularly urology drainage bags, that collect fluids during the surgical procedure, are known. There are reusable flexible band systems which connect with reusable clamps that mount to the distal ends of side rails of surgical tables. Reusable flexible bands allow mounting of a disposable drainage bag that then collects fluids during the procedure. There are also systems that consist of a disposable drainage bag mounted to a flexible band which in turn connects to the surgical table by means of a reusable rail mounted clamp. These reusable clamps are mounted at the distal end of the surgical table side rail. In the art there are also disposable drainage bag systems which consist of a disposable drainage bag mounted to a flexible band to which is attached disposable clamps which connect to the distal end of surgical table side rails. These disposable clamps will fit only one size of surgical table rails. The advantage of the completely disposable drainage bag system described above is that the clamps do not need to be cleaned and sterilized after each procedure and then stored for eventual re-use.

However, the clamps of such a system, and all conventional clamps used in the current art described in this application, occupy a portion of the distal end of the surgical table rail. According to the current art when disposable or reusable clamps of these systems are used with lithotomy stirrups the clamps that mount the stirrups must be moved proximally towards the patient's head since the disposable clamps are mounted at the distal end of the surgical table rail. Not mounting the lithotomy stirrups at the distal end of the surgical table rails may prevent the stirrup's proximal locking joint from being positioned at the patient's hip joint as recommended by all stirrup manufacturers.

If the proximal locking joint of the stirrup is not mounted at the patient's hip joint an increase in the stress on the patient's hip may occur, which may lead to possible injury or post procedural pain.

Instruments such scopes, retractors, forceps, often are used many times during procedures, such as procedures that require the patient to be placed in the lithotomy position with stirrups. These instruments can be secured by attachment clamps to the side rails of the surgical table. Since the distal end of the surgical table side rail may be occupied by clamps securing the stirrups to the surgical table, other instruments may be difficult to access in cases where the clinician is operating on the perineum.

### SUMMARY

This disclosure is generally directed to surgical drainage bag systems that are disposable (i.e., they can be discarded after each use). In some embodiments, such a surgical drainage bag system can include a disposable clamp, a disposable flexible band to which is attached a disposable drainage bag drape, all of which can be discarded after use. This system allows the user to attach the system to a surgical table and then discard the system after each use.

As discussed in detail below, a surgical drainage bag system according to the present teachings can allow the safe positioning of a patient since it allows the stirrup mounting clamp to be positioned at the distal end of the side rail of a surgical table. Where stirrups are not used, a surgical drainage bag system according to the present teachings allows for mounting the disposable clamps onto the distal end of the surgical table rails to secure the disposable drainage bag system.

Surgical table side rails to which accessories are attached, including drainage bag systems, come in a variety of sizes including differing widths and heights and include, but are not limited to US, EU and UK surgical table side rails. In the current art both the reusable clamps and disposable clamps of the conventional drainage bag systems will fit only one size of surgical table rails. This means that a facility with varying tables with rail sizes would have to stock a number of conventional types of drainage bag systems each with differing sized clamps. The current invention allows one disposable clamp to be engaged onto a variety of rail sizes meaning that hospitals with a variety of tables with differing rail sizes need procure only one type of drainage bag system.

In many urological and gynecological procedures, which require a fluid collection system, other accessories, equipment and surgical aids may be attached via surgical rail clamps to the side rails present on most surgical and procedural tables. Lithotomy stirrups are used to lift and hold the legs away from the operative site are but one example of commonly used surgical equipment accessories and/or surgical aids. Lithotomy stirrups when attached to the surgical table side rails via surgical rail clamps help provide the clinician access to the operative site. These lithotomy stirrups frequently employ gas filled cylinders which provide an upward force reducing the stress or strain on the clinician when lifting and positioning the patient's legs. The gas cylinders are typically attached at a distal mount point on the stirrup support rod and at a proximal mounting lug on a mount plate which is at about the equal level of the surgical table rail.

In some embodiments, a surgical drainage bag system according to the present teachings can include a flexible band and a disposable drainage bag drape along with a disposable clamp (which may be attached to the surgical table side rail), all of which can be discarded after each use plus an accessory mount mechanism that allows securing the disposable drainage bag, the flexible band and the clamp to equipment or accessories configured to be mounted onto a medical table, such as procedural or surgical tables.

In many embodiments, the disposable clamp allows the acceptance of a wide variety of sizes of surgical table rails obviating the need to stock multiple drainage bag systems with differing sized clamps to fit such differing rail sizes. The accessory mount mechanism may be attached to certain surgical accessories or equipment used during medical procedures in order to secure the disposable drainage bag system for use. Such an accessory mount for the disposable drainage bag system obviates the need for rail mounting the drainage bag system when certain surgical accessories are used, thus creating more space at the distal end of the surgical table rail. This additional space at the distal end of the surgical table rail may allow a lithotomy stirrup's proximal locking joint to be properly mounted at the location of a patient's hip joint, which in turn reduces the risk of creating stress at the patient's hip joint, that could lead to injury during the procedure and/or post procedural patient hip pain.

In embodiments, a disposable drainage bag system may include a disposable drainage bag, a flexible band coupled to and supporting the disposable drainage bag and a disposable clamp that is configured to accept a surgical table side rail or an accessory mount arm, which may be sized similar to a surgical table side rail.

The disposable clamp may include a channel to accept a surgical table rail, or an accessory mount arm, and a band acceptance slot, sized to accept the flexible band, on the other side of the disposable clamp. In some embodiments the band acceptance slot may have indentations and/or holes at pre-determined positions within the band acceptance slot that may be sized to receive detents, buttons, protuberances and protrusions that may be formed into the flexible band. In some embodiments, an upper ledge and a lower ledge may be formed into the disposable clamp body. These ledges may each have a stabilizer extension formed therein that are substantially rigid and substantially parallel to the lower surface and upper surfaces of the lower and upper ledges. The term “substantially rigid” is defined herein as deflecting less than 0.10 inch while substantially parallel means not deviating more than 10 degrees from the referenced surface. In some embodiments, the disposable clamp may be formed of a plastic, such as polyethylene, polyurethane, polycarbonate, polyethylene terephthalate, or acrylonitrile-butadiene-styrene (ABS).

In some embodiments a disposable clamp for coupling a flexible band and a drainage bag to a surgical table side rail or accessory mount arm is disclosed. The disposable clamp may include a rail acceptance channel on one side thereof for accepting the side rail of a surgical table or an accessory mount arm. In some embodiments the rail acceptance channel may be formed by the upper ledge and lower ledge of the disposable clamp while each ledge may have lips formed into the distal edges of the upper and lower ledges of the disposable clamps. The clamp may have a body including a proximal side in some embodiments. In some embodiments, the proximal side of the clamp may have a notch cut or formed therein. The acceptance rail channel may be shaped and sized to fit onto a surgical table rail or an accessory mount arm.

In some embodiments the lower surface of the upper ledge may have a chase cut into it allowing the acceptance of differing sizes of accessory rails; in particular rails that may have a width narrower, or a height larger, than the rail acceptance channel of the disposable clamp. In other embodiments upper ledge chase can fit onto at least one fin formed into accessory mount arms to stabilize the disposable clamp when so mounted. In some embodiments, the lower ledge can be formed to include a threaded hole that can receive a threaded knob which can be engaged into and through the threaded hole. In some embodiments the threaded hole is offset from the center of the clamp body and is aligned on the centerline of the chase formed into the lower surface of the upper ledge of the disposable clamp. In some embodiments, a threaded screw can be engaged through a threaded hole in lower platform and a surgical rail may be inserted into upper ledge chase thus engaging the surgical table rail with upper ledge chase. In some embodiments the lower surface of the lip of the upper ledge is lower than the notch cut onto the upper ledge of the proximal clamp surface forming one side of the rail acceptance channel. In other embodiments, a threaded screw can be engaged through a threaded hole in the lower platform engaging the clamp with an accessory mount arm. In other embodiments, the upper ledge may be formed to include a threaded hole that can receive a threaded knob which can be engaged into and through the threaded hole. In some embodiments, the lower ledge may include a distal lip and also include an extension stabilizer that protrudes from the lower ledge opposite the side of the disposable clamp's band acceptance slot. In one embodiment the upper ledge of the clamp may include a lip on its distal side and also may have an engagement surface, which is lower than a notch in the proximal clamp wall and may also have a stabilizer extension that protrudes from the ledge on the side opposite the disposable clamp's band acceptance slot. In such an embodiment, the lips formed on the upper ledge may protrude distally and down and away from the upper surface of this ledge to assist in engaging the surgical table rail or accessory mount arms. In some embodiments the lips formed on the lower ledge may protrude distally and up and away from the lower surface of the lower ledge to assist in engaging the surgical table rail or accessory mount arm. In some embodiments, the proximal side of the clamp body with notch, the lower upper ledge and the lower ledge, the upper and lower edge lips and the upper and lower stabilizer extensions of the ledges may form a monolithic body formed, e.g., of plastic.

The proximal side of the clamp body with notch, the lower surface of upper ledge, the lips of the upper ledge including an engagement surface and the lower ledge with lips, and the upper and lower stabilizer extensions of the ledges make up the rail acceptance channels into which a surgical table rail or accessory mount arm may be engaged. In some embodiments the lower surface of the upper ledge may have a chase cut into it to accept rails that are narrower and/or taller than the width of the rail acceptance channel. In other embodiments the components of the surgical rail acceptance channel may include separate plastic or metal pieces or a combination of metal and plastic parts that can be assembled, e.g., via press fit, welded or glued together. In yet other embodiments the disposable clamp body, ledges, lips and extension stabilizers can be formed of metal.

The disposable clamp may be engaged onto a table surgical table rail or accessory mount arm using the clamp rail acceptance channel. In some embodiments the upper portions of surgical table rails are narrower and/or taller than rail acceptance channel and can fit into the upper ledge chase. In other embodiments the upper surface chase can be fitted onto a fin formed into upper surface of an accessory mount arm. The threaded knob can then be inserted and engaged into the threaded hole in the lower ledge such that the distal portion of the threaded knob in turn engages the surgical table rail or an accessory mount arm thus securing the disposable clamp in place. In other embodiments the threaded knob can be engaged into, and through, the threaded hole pushing against rails narrower and/or taller than rail engagement channel and engaging them into the upper ledge chase thus securing this rail to the disposable clamp.

In some embodiments the disposable clamp side opposite the rail acceptance channel may have a flexible band acceptance slot formed in it. The flexible band acceptance slot may have at least one indentation in one embodiment. The at least one indentation can have a variety of shapes including, but not limited to, rectilinear, square, cuboidal, round, oblong, hexagonal, round or obolid or formed in other shapes. In other embodiments the clamp rail channel may have at least one through hole. The at least one through hole can have a variety of shapes including, but not limited to rectilinear, square, cuboidal, round/circular, obolid, oblong or hexagonal. In other embodiments the band acceptance slot can have at least two indentations in it and each of the indentations can be of the same or differing shapes or sizes including, but not limited to, the following shapes: rectilinear, square, cuboidal, round/circular, obloid, oblong or hexagonal. In other embodiments the disposable clamp band acceptance slot may have at least two through holes. The at least two through holes can have a variety of shapes including, but not limited to the following: rectilinear, square, cuboidal, round/circular, obloid, oblong or hexagonal.

In some embodiments the clamp side opposite the rail acceptance channel may have a band acceptance slot formed in it. The band acceptance slot may have at least one indentation in one embodiment. The at least one indentation can be formed to accept protuberances or protrusions that are serrated or barbed or other polygonal shapes. In other embodiments the clamp rail channel may have at least one through hole or indentation. The at least one through hole or indentation can be formed to accept protuberances that are serrated or barbed protrusions or of other polygonal shapes. In other embodiments the clamp band slot can have at least two indentations or through holes in it and each of the at least two through holes or at least two indentations can be of the same or differing shapes or sizes in any combination that might accept barbed or serrated protuberances or other polygonal shapes. In other embodiments the at least one through hole or indentation can be formed to accept a variety of shapes including but not limited to barbed or serrated shaped protrusions.

In some embodiments the clamp side opposite the rail acceptance channel may have a band acceptance slot formed in it. In some embodiments the band acceptance slot may have at least one indentation and at least one through hole. The at least one indentation and the at least one through hole can be formed to accept protuberances that are serrated or barbed protrusions or otherwise shaped and in any combination of shapes. The clamp band slot can have at least one indentation and at least one through hole in it and each of the at least one through hole or the at least one indentation can be of the same or differing shapes or sizes in any combination that might accept barbed or serrated protuberances or shapes in any combination.

In some embodiments the surgical drainage bag system can include a disposable drainage drape bag, a flexible band, a disposable clamp and an accessory mount mechanism. This accessory mount mechanism may be secured to an accessory or other equipment used in surgical procedures, in particular lithotomy stirrups, which are configured to be attached to a procedural or surgical table in some embodiments. In one embodiment the accessory mount mechanism can be discarded after use. In other embodiments the accessory mount mechanism may be semi-permanently attached to an accessory or other equipment configured to attach to a procedural or surgical table in particular lithotomy stirrups. This accessory mount mechanism may be formed or molded of plastics parts in one embodiment. In other embodiments it may be formed or made of metal. In yet other embodiments it may be made or formed of a combination of plastic and metal parts. It is contemplated in some embodiments that the accessory mount mechanism may be formed as a monolithic part of either plastic or metal. In other embodiments it can be made of two or more separate plastic or metal pieces that may be press fit using indentations and counter-pressed protuberances or protrusions or held together by other mechanical means such as an attachment rod. In some embodiments the accessory mount mechanism may include a monolithic accessory mount clamp formed of plastic or of metal with at least two holes formed in the structure and with at least one of the holes being threaded, plus a separate spacer formed of plastic or made of metal with a through hole and an attachment rod threaded at one end with a flat head on the opposite end.

The accessory mount mechanism, in some embodiments, may include a horseshoe or “u” shaped accessory mount clamp with holes formed in two of its arms, a spacer with a through hole and an attachment rod threaded at one end with a flat head at its opposite end. In some embodiments the spacer is “t” shaped while in other embodiments it is shaped as a rectilinear block. In yet other embodiments it can be round or semi-circular in shape. The accessory mount clamp is shaped and sized so that it fits snugly and closely around the outside surfaces of a stirrup gas cylinder mount lug of a lithotomy stirrup while the spacer is attached to the mount accessory clamp. There are differing sized proximal mount lugs for gas cylinders for various makes of stirrups. In some embodiments, there are differing sized accessory mount clamps and spacers that will accommodate and fit closely and snuggly around the outside surfaces of various sizes of gas cylinder gas mount lugs for each make of stirrup.

In some embodiments the accessory mount clamp can include an upper arm, a lower arm and a semi-circular section. The upper arm, the lower arm and the semi-circular section of the accessory mount clamp is designed to fit closely and snuggly with the outside surface of the stirrup gas cylinder mount lug. In some embodiments the upper arm, lower arm and semi-circular sections of the accessory mount clamp are formed or molded as a monolithic part out of plastic or alternatively formed or made of metal. In yet other embodiments the sections are plastic or metal and are press fit or glued together using indentations with slots to accept barbed or serrated protrusions or protuberances. In one embodiment the upper arm is formed with a threaded hole that can accept a threaded rod while the lower arm is formed with a hole that is opposite the corresponding hole in the upper arm. In other embodiments the lower arm has a threaded hole while the upper arm has a non-threaded hole which is directly opposite the hole of the upper arm. In some embodiments a mount arm is formed and centered on the distal edge of the semi-circular section and protrudes outward at substantially right angles from this section. The term substantially right angles is defined as plus or minus 5 degrees from 90 degrees. The mount arm is the size of a surgical table rail. In some embodiments a fin which is sized to fit into the upper ledge chase of disposable clamp is formed into the upper surface of accessory mount arm. A spacer formed or molded from plastic or made from metal, almost equal in length to twice the inner radius of the semi-circular section of the accessory mount clamp in some embodiments, is made or formed of plastic or of metal. The spacer has a through hole running from its top through to its bottom in some embodiments. The spacer fits on part of the surface of the stirrup gas spring mount lug and the through hole formed in the spacer that lines up with the holes formed in the upper and lower arms of the accessory mount clamp. In one embodiment an attachment rod (machined or formed of metal, plastic or suitable materials) with one threaded end and a flat head on the opposite end is passed through the lower arm of the accessory mount clamp and into and through the through hole in the spacer. The threaded end of the rod is designed to fit into, and engage with, the threaded hole in the upper arm of the accessory mount clamp in one embodiment.

In some embodiments a spacer rod (machined or formed of metal) with one threaded end and a flat head on the opposite end is passed through the hole in the upper arm of the accessory mount clamp into and through the through hole in the spacer. The threaded end of the rod is engaged into and with the threaded hole in the lower arm of the accessory mount clamp thus securing the accessory mount clamp to the surfaces of stirrup gas cylinder mount lug. In some embodiments the spacer is rectangular in shape. In other embodiments it is “T” shaped with a flange. In some embodiments the “T” shaped flange has side having a semi-circular cut while in other embodiments this “T” flange has a rectilinear cut out. In other embodiments the spacer is circular, obloid or semi-circular in shape and may have a flange of varying shapes including but not limited to square, circular or hexagonal. The spacer, secured by the threaded rod to the upper and lower arms of the accessory mount clamp, also fits snuggly to a surface of the stirrup gas cylinder mount lug.

It is contemplated that mount arm with fin in upper surface that protrudes from the distal side of the semicircular section of the accessory mount clamp is formed or molded as part of the monolithic plastic or metal structure of the accessory mount clamp in one embodiment. In other embodiments the mount arm and fin may be formed of separate pieces of plastic or metal and connected to the accessory mount clamp by means of press fit connections or gluing/welding or any combinations thereof. The mount arm can be sized to conform to the size of a surgical table rail in some embodiments.

In some embodiments the accessory mount clamp may be attached to the stirrup gas cylinder mount lug by fitting the accessory mount clamp's upper arm, lower arm and semi-circular sections around the surface or surfaces of stirrup gas cylinder mount lug. By passing the attachment rod through the upper arm of the accessory mount clamp and into and through the through hole in the spacer it can be engaged into the threaded hole of the lower arm of the accessory mount clamp thus securing the spacer to the accessory mount clamp and in turn securing the accessory mount clamp to the stirrup gas cylinder mount lug. The side of the disposable clamp with the rail acceptance channel can be slid onto the mount arm with fin of the accessory mount clamp with the upper ledge chase fitting round the mount arm fin. The upper and lower stabilizer extension arms of the disposable rail clamp and the fit of the mount arm fin onto upper ledge chase help stabilize the disposable clamp resisting any torsion forces. The threaded knob of the disposable clamp can be engaged into the threaded hole in lower ledge of the disposable clamp securing it to the mount arm of the accessory mount clamp in some embodiments. The flexible band with the attached drainage bag drape can then be secured to the disposable clamp.

The accessory mount mechanism, in some embodiments, may include a horseshoe or “u” shaped accessory mount clamp with upper, lower and curved arms with holes formed in its upper and lower arms, a spacer with a through hole and an attachment rod threaded at one end with a flat head at its opposite end. In some embodiments, the spacer is “t” shaped while in other embodiments it is shaped as a block. In yet other embodiments it can be round or semi-circular in shape. In some embodiments the spacer may have a rectilinear or circular flange and the flange may have a semi-circular or rectilinear cutout. The accessory mount clamp is sized so that it fits snugly and closely around the surface or surfaces of a protrusion or extension of a surgical accessory or surgical equipment with such extensions being between about 0.25 to about 4 inches in diameter (for circular protrusions) or longest cross section width (in rectilinear protrusions) being between about 0.25 to about 4 inches and protrusions extending out from ½ inches up to eight inches in length from the surface of the equipment or accessory in some embodiments. The spacer, when attached to the mount accessory clamp fits, snuggly around the proximal surfaces of the protrusion of the surgical accessory or equipment when such a protrusion is between 0.25 to 4 inches in diameter or cross sectional width of from about 0.25 to about 4 inches and extending outward from the surface of the accessory or equipment up to eight inches in length in some embodiments.

In other embodiments there may be a mount lug or protrusion that is formed into a surgical table accessory rail clamp which is used to attach accessories to a surgical table. The rail clamp mount lug can be sized so that the accessory mount clamp may be fitted closely and snuggly around the outside of this lug. In some embodiments the accessory mount clamp may be attached to the rail clamp mount lug of the surgical table rail accessory clamp by fitting the accessory mount clamp's upper arm, lower arm and semi-circular sections around the surface or surfaces of stirrup gas cylinder mount lug. By passing the attachment rod through the upper arm of the accessory mount clamp and into and through the through hole in the spacer it can be engaged into the threaded hole of the lower arm of the accessory mount clamp thus securing the spacer to the accessory mount clamp and in turn securing the accessory mount clamp surgical table rail clamp mount lug. The side of the disposable clamp with the rail acceptance channel can be slid onto the mount arm with fin of the accessory mount clamp with the upper ledge chase fitting round the mount arm fin. The upper and lower stabilizer extension arms of the disposable rail clamp and the fit of the mount arm fin onto upper ledge chase help stabilize the disposable clamp resisting any torsion forces. The threaded knob of the disposable clamp can be engaged into the threaded hole in lower ledge of the disposable clamp securing it to the mount arm of the accessory mount clamp in some embodiments. The flexible band with the attached drainage bag drape can then be secured to the disposable clamp.

In some embodiments a surgical rail clamp may have a mount arm with a fin formed into its distal end. The side of the disposable clamp with rail acceptance channel can be slid onto the mount arm with fin of the accessory mount arm with the upper ledge chase fitting round the mount arm fin. The upper and lower stabilizer extension arms of the disposable rail clamp and the fit of the mount arm fin onto upper ledge chase help stabilize the disposable clamp resisting any torsion forces. The threaded knob of the disposable clamp can be engaged into the threaded hole in lower ledge of the disposable clamp securing it to the mount arm of the accessory mount arm in some embodiments. The flexible band with the attached drainage bag drape can then be secured to the disposable clamp.

In some embodiments a surgical table accessory rail clamp may have a mount arm with a fin formed into with the mount arm protruding distally. In other embodiments the mount arm with a fin formed into it may be affixed through mechanical attachments to the surgical table accessory rail clamp. The side of the disposable clamp with the rail acceptance channel can be slid onto the mount arm with fin of the accessory mount arm with the upper ledge chase fitting round the mount arm fin. The upper and lower stabilizer extension arms of the disposable rail clamp and the fit of the mount arm fin onto upper ledge chase help stabilize the disposable clamp resisting any torsion forces. The threaded knob of the disposable clamp can be engaged into the threaded hole in lower ledge of the disposable clamp securing it to the mount arm of the accessory mount arm in some embodiments. The flexible band with the attached drainage bag drape can then be secured to the disposable clamp.

The flexible band may be formed of commonly available plastic or other suitable materials. The flexible band may have at least one prong formed into each of its ends in one embodiment. The at least one prong may have at least one protrusion or protuberance that can be formed into differing shapes. In some embodiments the at least one protuberance or protrusion in the prong may be, but is not limited to, the following shapes: rectilinear, square, cuboidal, round, obolid, oblong, hexagonal or round in any combination but sized to fit in the at least one indentation in the flexible band acceptance slot. In other embodiments the shapes of the at least one protuberance or protrusion in the at least one prongs may be serrated or barbed in shape and in any combination but sized to fit into the at least one indentation in the flexible band acceptance slot. In some embodiments the flexible band may have at least one notch cut into its ends forming at least one prong with at least one protuberance on the prong of the flexible band.

The flexible band may be formed of commonly available plastic or other suitable materials. The flexible band may have at least two prongs formed into each of its ends in some embodiments. The at least two prongs may have at least one protrusion or protuberances each that can be formed into differing shapes. In some embodiments the at least one protuberance or protrusion formed in the at least two prongs may be, but is not limited to, the following shapes: rectilinear, square, cuboidal, round, obolid, oblong, hexagonal or round in any combination but sized to fit in the at least two indentations in the flexible band acceptance slot. In other embodiments the shapes of the at least one protuberance or protrusion in the at least two prongs may be serrated or barbed in shape and in any combination but sized to fit into the at least two indentations in the flexible band acceptance slot. In some embodiments the flexible band may have at least one notch cut into its ends forming at least two prongs with at least one protuberance on the at least two prongs of the flexible band.

In some embodiments the distance between the at least one protuberance on the differing prongs of the flexible band is slightly larger than the height of the disposable band acceptance slot formed into the side of the disposable clamp. The term “slightly large” is defined herein as being between about 0.1 to about 0.15 inches larger. When the flexible band is fitted into the disposable band acceptance slot the at least two prongs are depressed by the force exerted on the at least two prongs as they are engaged into the band acceptance slot. As the end of the flexible band is slid into the acceptance band slot the at least two protuberances engage with the at least two indentations or through holes in the flexible band acceptance slots thus connecting the flexible band with the disposable clamp.

The flexible band may be formed of commonly available plastic or other suitable materials in one embodiment. At least one notch may be cut into the ends of the flexible band forming at least two prongs. Each of the at least two prongs may have at least one protrusion or protuberance formed into them in one embodiment. The at least one protrusion or protuberance of each of the at least two prongs may be of differing shapes and are formed into the at least two prongs at each end of the flexible band. In other embodiments the shapes of the at least one protuberance or protrusion on each of the at the least two prongs may be serrated or barbed in shape or any suitable shape but sized to fit into the at least two through holes or indentations in the clamp band slot. In other embodiments two notches are cut into the end of the disposable band forming upper, middle and lower prongs with the at least two prongs having at least one protuberance each. The at least one protuberance on each of the at middle and top prongs, in some embodiments, are sized to engage with the at least two indentations or at least two through holes in the band acceptance slot formed in the side of the disposable clamp body.

The maximum distance between the at least one protuberance on the upper prong and the at least one protuberance on the lower prong is slightly larger than the height of the band acceptance slot. Slightly larger is defined as being between about 0.01 and about 0.15 inches larger. When the flexible band is engaged into the disposable band acceptance slot the at least two prongs are depressed as they are engaged into the band acceptance slot. As the end of the flexible band is slid into the acceptance band slot the at least one protuberance engages with the at least two indentations or at least two through holes in the flexible band acceptance slots, thus connecting the flexible band with the disposable clamp.

In some embodiments, the disposable clamp can be molded or formed from plastic, metal, or both as a monolithic part. In other embodiments the clamp can be assembled from separate pieces with such pieces being pressed fit together using barbed or serrated indentations and protrusions. Such separate pieces could also be glued or welded together.

The drainage bag drape can be made of a flexible waterproof polymer and can be circular or semi-circular in shape in some embodiments. In some embodiments the drainage bag drape is clear with the upper portion of the drainage bag drape coupled to the flexible band that can be in turn attached to the disposable clamp. The disposable clamp can be attached to a surgical table rail or alternatively attached to the mount arm of the accessory mount clamp. The drainage bag drape can be “v” shaped and larger at the top than at the bottom with the bottom of the drape ending in a hole with a threaded tube attached to it. In some embodiments, the threaded tube can be attached by mechanical means or by gluing means to the hole in the bottom of the drape. The threaded tube can be attached to another threaded tube which in turn is connected to a fluid collection system which stores the fluid. In some embodiments the drape has a filter between the top of the drape and its bottom. The filter can be in the form of a mesh, which in turn can be made, for example, of cross woven thread of flexible polymer materials.

In a related aspect, a drainage bag system configured to be coupled to a medical procedural table is disclosed, which includes a drainage bag, a band attached to and supporting the drainage bag, said band having at least one protuberance positioned at least one end thereof, and at least one mounting clamp including a receiving slot configured to receive said at least one protuberance of the band. The clamp has a receiving channel that is configured to receive a procedural table accessory rail or an accessory mount arm thereof. The clamp has a threaded member configured to secure the clamp to a procedural table accessory or an accessory mount arm.

In some embodiments of the above drainage bag system, the at least one protuberance includes two protuberances each located at one of the two ends of the band and the at least one mount clamp includes two mount clamps each configured to receive one of those protuberances.

In some embodiments, the receiving slot is configured to provide at least one receptacle extending into a body of the mounting clamp for receiving said at least one protuberance so as to block the band from disengaging from the receiving slot. In some such embodiments, the at least one mounting clamp includes an upper platform, a lower platform and a clamp wall. The upper platform can include an upper protruding lip and the lower platform can include a lower protruding lip. The mounting clamp can further include at least one upper arm that extends from a proximal side of the upper platform and at least one lower arm that extends from a proximal side of the lower platform.

In some embodiments, the upper and the lower arms are substantially parallel with the surfaces of the upper and the lower platforms. In some embodiments, the lower platform comprises a threaded hole and a threaded knob to engage in the hole. The upper platform can also include a threaded hole and a threaded knob to engage in the hole.

In some embodiments, the upper platform includes a threaded hole and a threaded knob to engage a rail accessory channel formed by a lower surface of the upper platform and the protruding lip thereof, the upper surface of said lower platform and the protruding lip thereof, the arm extending from the upper and lower platforms, and the at least one side of the clamp body.

In some embodiments, the lower surface of the upper platform comprises at least one chase or channel formed therein. In some such embodiments, the at least one chase can have a width in a range from about 0.9 inches to about 1.6 inches and a height in a range of about 0.05 inches to about 0.25 inches.

The rail accessory channel is shaped and sized to receive a surgical table accessory rail or an accessory arm. In some embodiments, the rail accessory channel has a chase cut or formed therein, where the chase is configured to accept a protrusion extending out of the upper surface of an accessory mount arm. In some embodiments, the protrusion includes a fin. In some embodiments, the upper platform with the chase and the protruding lip, the lower platform and its protruding lip, the extension arms, the clamp wall, and clamp body are formed as a monolithic unit. By way of example and without limitation, such a monolithic unit can be formed of a plastic (e.g., polyethylene, polyurethane, polycarbonate, polyethylene terephthalate, or acrylonitrile-butadiene-styrene (ABS), a metal (e.g., aluminum, stainless steel, cast iron, titanium, or brass or other copper alloys), or a combination thereof.

In some embodiments, the upper platform with the chase and the protruding lip, and the lower platform and its associated protruding lip, the extension arms, the clamp wall, and the clamp body are formed as a combination of discrete parts, e.g., metal parts, plastic parts, or a combination of metal and plastic parts, that have been assembled, e.g., via press fit and/or glue and/or welding.

In some embodiments, the band of a drainage bag system according to the present teachings is configured to be received via a protrusion thereof into a pre-determined position within the clamp so as to engage with the clamp. In some embodiments, the band can have a width in a range of about 0.5 inches to about 3 inches, though other widths may also be utilized. Further, in some embodiments, the receiving channel of the clamp can have a width in a range of about 0.2 inches to about 0.45 inches and a height in a range of about 0.9 inches to about 1.6 inches.

In a related aspect, a drainage bag system configured to be coupled to a device which is configured to be coupled to a procedural table or other equipment is disclosed, which comprises a drainage bag, a band attached to and supporting the drainage bag, a mounting clamp including a receiving slot configured to receive an end of the band, where the band has at least two opposed protuberances at said end thereof configured to extend into the receiving slot and the receiving slot includes a pair of receptacles configured to receive the band protuberances for engaging the band with the mounting clamp. The clamp further includes a receiving channel to receive any of an accessory rail and an accessory mount arm of said procedural table.

In some embodiments, the clamp can include a threaded member for securing the clamp to the procedural table.

In some embodiments, the accessory rail and/or the accessory mount has a width in a range of about 0.2 inches to 0.45 inches and a height in a range of about 0.9 and 1.6 inches.

The drainage bag system can further include a device mount mechanism configured to secure the drainage bag system to any of an accessory, and a device that is coupled to the procedural table. In some embodiments, the accessory mount mechanism can include at least one arm that is shaped and sized to fit into the receiving channel.

Additional features, alone or in combination with these and any other feature(s); such as those listed above and those listed in the claims may comprise patentable material.

In other embodiments, the attachment rod can be passed through the upper arm of the accessory mount clamp and into and through the hole in the spacer and then passed through a hole in the lower arm of the accessory mount clamp. In this embodiment, the attachment rod can have a cap on the upper end of the rod and a hole in its lower end through which a cotter key can be placed securing the spacer to the accessory mount clamp and in turn securing the accessory mount clamp to the gas cylinder. In yet another embodiment, the upper end of the attachment rod can have a cap on it and be passed through the upper arm of the accessory mount clamp and the into and passed through the hole in the lower arm of the accessory mount clamp. A cap can then be welded, glued or mechanically attached to the lower end of the attachment rod securing the spacer to the accessory mount clamp.

Some embodiments are directed to a mount configured to attach to a mount lug, the mount comprising accessory mount clamp comprising a mount arm extending from the accessory mount clamp, and a spacer comprising a hole extending through the spacer through which a rod can be passed to secure the spacer to the accessory mount clamp, thereby connecting the accessory mount clamp to the mount lug.

Some embodiments are directed to a mount configured to attach to a mount lug, the mount comprising, accessory mount clamp comprising mount arm extending from the accessory mount clamp; and a fastener to connect the accessory mount clamp to the mount lug.

The accessory mount clamp can comprise an upper arm having a hole formed therein, semicircular section, and a lower arm having a hole formed therein.

The proximal mount lug can be disposed on a stirrup bracket and the spacer can includes a semi-circular cut-out that is configured to mate with a proximal surface of the mount lug.

In some embodiments, the mount arm is configured to attach to a disposable clamp to which an accessory can be mounted. In some embodiments, disposable clamp can include a rail acceptance channel that is configured to engage with the mount arm.

Some embodiments are directed to a mount comprising a cap that attaches to the upper end of rod to secure the rod to accessory mount clamp.

Some embodiments can include a cotter key that is configured to extend through hole formed in the rod to secure the accessory mount clamp to the spacer, thereby securing the accessory mount clamp to the proximal mount lug, or a securement pin that secures the cotter key to the rod.

In some embodiments the accessory mount clamp is a monolith or made of discrete parts.

Some embodiments are directed to a mechanism for attaching a first accessory to a second accessory attached to a surgical table comprising an accessory mount clamp configured to engage with a proximal mount lug, the accessory mount clamp having a hole formed therein, a mount arm disposed adjacent to the accessory mount clamp, a spacer connectable to the accessory mount clamp and a rod configured to pass through holes formed in an upper portion and lower portion of the accessory mount clamp and configured to pass through holes formed in the spacer thereby connecting the spacer and the accessory mount clamp around the proximal mount lug.

In some embodiments, the second accessory is a leg holder accessory and the first accessory is a surgical bag.

In some embodiments, the mount arm is configured to attach to a disposable clamp to which the accessory can be mounted.

In some embodiments, the disposable clamp includes a rail acceptance channel that is configured to engage with the mount arm.

Some embodiments are directed to a mechanism for attaching a first accessory to a second accessory attached to a surgical table comprising an accessory mount clamp configured to engage with a proximal mount lug, the accessory mount clamp having a hole formed therein, a mount arm disposed adjacent to the accessory mount clamp, and a fastener to connect the accessory mount clamp to the mount lug.

In some embodiments, the second accessory is a leg holder accessory, or the first accessory is a surgical bag.

In some embodiments, the mount arm is configured to attach to a disposable clamp to which the accessory can be mounted, or the disposable clamp can include a rail acceptance channel that is configured to engage with the mount arm.

Some embodiments are directed to an accessory clamp configured to be secured to a surgical table rail of a surgical table comprising an accessory slot configured to receive a mounting member, a channel formed therein configured for attaching to the surgical table rail, and an accessory mount arm formed into distal surface of the accessory clamp to which an accessory can be mounted.

In some embodiments, the extended portion comprises a fin, or the accessory mount clamp comprises an upper arm, lower arm, circular portion and rod configured to secure the adapter to the mount lug.

Some embodiments are directed to an accessory clamp configured to be secured to a surgical table rail of a surgical table comprising an accessory slot configured to receive a mounting member, a channel formed in the accessory clamp configured for attaching to the surgical table rail, and a mount arm attached to the accessory clamp and configured to receive a disposable clamp mounted thereto.

In some embodiments, the mount arm comprises an extended portion, or the extended portion comprises a fin.

In some embodiments, the mount arm comprises a cutaway portion for receiving an upper portion of the mount arm, or the mount arm comprises holes formed therein for receiving bolts that are configured to secure the mount arm to the accessory clamp.

In some embodiments, the mount arm comprises a knob configured to secure the mounting member to the accessory clamp.

In some embodiments, the accessory mount clamp extends at least partially around the proximal mount lug, or the accessory mount clamp extends at least partially around the proximal mount lug.

In some embodiments, the accessory clamp can include a lug and an accessory mount clamp configured to attach to the lug, wherein the accessory mount adapter comprises an extended portion configured to accept an accessory.

In some embodiments, the mounting member is an accessory blade.

### BRIEF DESCRIPTION OF DRAWINGS

Drawings and figures are disclosed to aid in a description of invention and the various embodiments of the invention. The drawings and figures are attached and are referenced below.

FIGS. 1A and 1B illustrate views of a stirrup mounted onto a surgical table with a conventional drainage bag system and without such a system, respectively.

FIGS. 2A and 2B illustrate views of a disposable drainage bag system according to an embodiment of the present teachings, which is mounted onto a surgical table.

FIG. 2C is a partial perspective view of various components the disposable drainage bag system mounted on a surgical table with rail and a fluid storage system.

FIG. 3 shows a perspective of the disposable band with disposable drainage bag drape, disposable clamp and details of the reusable accessory mount clamp.

FIG. 4 shows another perspective view of the disposable band with disposable drainage bag drape, disposable clamp and details of the reusable accessory mount clamp.

FIG. 5 shows a perspective view of the disposable band with disposable drainage bag, disposable clamp and the details of reusable accessory mount clamp.

FIG. 6 shows details of a stirrup mount lug, the disposable clamp, the disposable band and the accessory mount clamp.

FIG. 7 shows another view of details of a stirrup mount lug, the disposable clamp, the disposable band and the accessory mount clamp.

FIG. 8 show details of the disposable clamps, the disposable band and the surgical table rail.

FIG. 9 illustrates details of an accessory mount mechanism coupled to a lug protruding from medical equipment in particular a stirrup mounting bracket.

FIG. 10 illustrates a cut away view of a section of the disposable clamp, the band acceptance slot, the flexible band with flexible prongs engaged in through holes within the band acceptance slot the disposable rail clamp engaged with an accessory mount arm.

FIGS. 11A and 11B illustrate cutaway sections of a disposable clamp and a rail acceptance channel of a system according to an embodiment of the present teachings.

FIG. 12 illustrates an embodiment in which a mount is attached to stirrup gas cylinder proximal mount lug.

FIG. 13 illustrates an embodiment in which a mount is attached to stirrup gas cylinder proximal mount lug.

FIG. 14 illustrates an exploded view of one embodiment where a mount is attached to a mount lug formed into an accessory clamp.

FIG. 15 illustrates an embodiment of a mount arm for a mount is attached to an accessory clamp.

FIG. 16 illustrates a mount arm for a mount arm is attached to an accessory clamp.

FIGS. 17A-C illustrate a side view, cross sectional view and a top view of a bladed accessory (a leg holder/stirrup) mounted via an accessory clamp to the side rail of a surgical table.

### DETAILED DESCRIPTION

The present disclosure is generally related to a drainage bag system configured for coupling to a surgical table. Various terms are used herein in accordance with their ordinary meanings in the art. The term “about” as used herein is intended to indicate a variation of at most 10% about a numerical value. The term “surgical table” is used herein to generally refer to a medical table that is used when a medical procedure, such as a urological or gynecological procedure, is performed on a patient.

The phrase “a semi-rigid component” is defined as a member or component so formed or constructed that when a force is applied to its center the elongation of the member's non-loaded length (change in length) is greater than 0% but less than 0.10% before failure.

The phrase “a flexible component” is defined as a component or member so formed or constructed that when a force is applied to its center the elongation of the member's non-loaded length is equal to or greater than 0.10%.

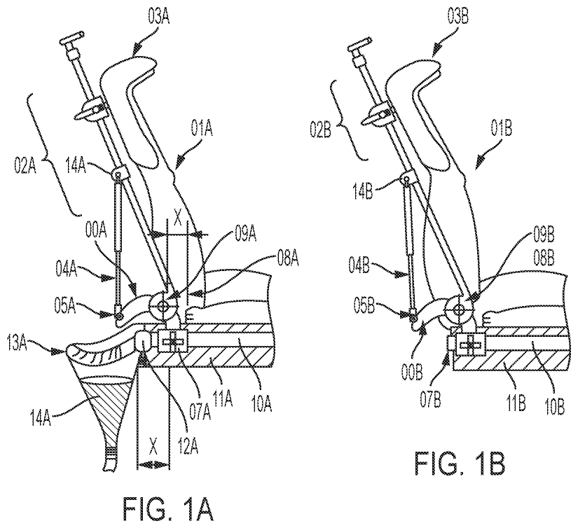

FIGS. 1A and 1B illustrate a stirrup mounted onto a surgical table without a drainage bag system and with a drainage bag system using conventional systems. A stirrup 02A of FIG. 1A is mounted to a surgical table 11A by a rail clamp 07A. Patient legs 01A are held in place by stirrup boot 03A which are supported by stirrup gas cylinder 04A attached to the stirrup at distal stirrup mount point on 14A and proximal stirrup gas cylinder mount lug 05A attached to stirrup bracket 00A. As shown in FIG. 1A, disposable clamp 12A holds flexible band 13A onto which is attached a drainage bag 14A. The mount point on the surgical table rail 10A of disposable clamp 12A can move mount point of table clamp 07A and stirrup 02A proximally down surgical table rail 10A, towards the patient's head, a distance equal to “x”. The center of rotation of the proximal stirrup locking joint 09A in FIG. 1A is offset from center of rotation of point of patient's hip 08A. This offset is caused by the mounting of disposable rail clamp 12 on distal end of surgical table rail 10A at distance “x.” FIG. 1B illustrates mounting of stirrups without using current art disposable drainage bag system. In FIG. 1B the rotation point of the patient's hip 08B and the rotation point of the proximal stirrup locking joint 09B are coterminous in the horizontal plane and thus in alignment due to rail clamp 07B being properly positioned at the distal end of the table rail 10B of surgical table 11B. Patient's legs 01B are held in the stirrup boot 03B of stirrup 02B and supported by stirrup gas cylinder 04B with distal stirrup mount points 14B and proximal stirrup gas spring mount lug 05B attached to stirrup mount bracket 00B.

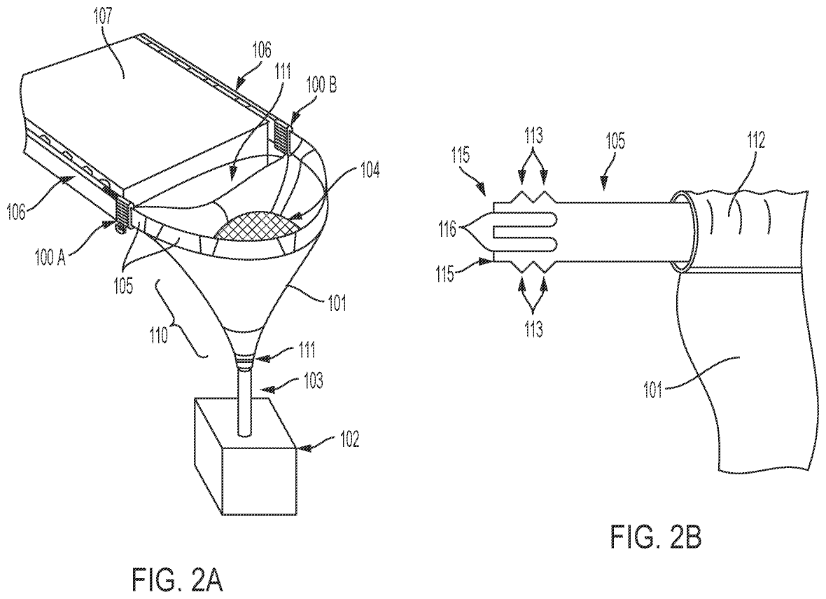

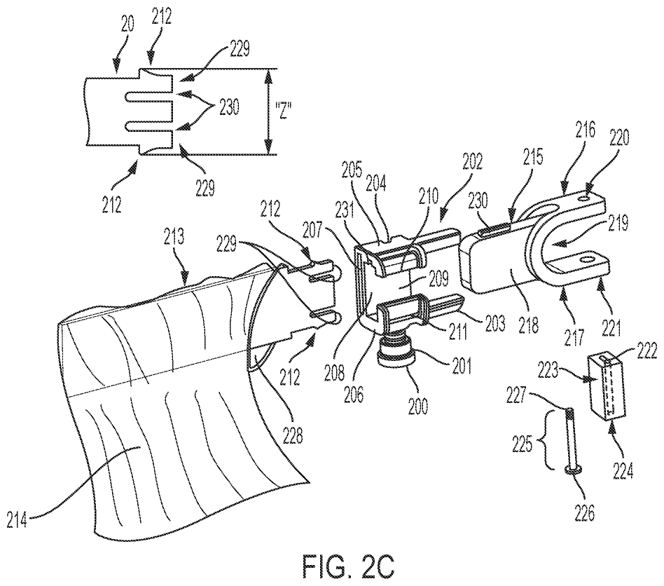

FIGS. 2A and 2B schematically illustrate a disposable drainage bag system 110 according to one embodiment of the present teachings, which is attached to a surgical table 107 side rails 106. The drainage bag system 110 includes a flexible band 105 to which the drainage bag 101 having a mesh filter 104 is attached, a first disposable clamp 100A and a second disposable clamp 100B which are shown as attached to the distal ends of surgical table side rails 106. Attached to the drainage bag 101 is a drainage tube 103 and fluid storage system 102. A drainage bag drape 110 is supported and kept open by flexible band 105 during surgery. Flexible band 105 is coupled to disposable clamps 100A and 100B which can in turn be attached to the distal ends of side rails 106 of surgical table 107. Threaded tube 103 attached to fluid reservoir 102 is engaged with and attached to flap 111 to allow fluid to flow from the drainage bag 101 into fluid storage system 102.

The illustrated drainage bag 101 is constructed from flexible sheets of thin translucent plastic materials and has a flap 111 attached to it. In this embodiment, drainage bag 101 is substantially V-shaped with the mesh filter 104 constructed out of thin flexible strips of a polymeric material laid in alternating or crossing patterns and includes a flap 111 attached at a hole 101a (not shown) at bottom of drainage bag 101 to allow fluid to drain out of the system and into a reservoir 102. Mesh filter 104 collects waste materials that may be by-products of surgical procedures and if not trapped in the mesh filter could prevent fluid from being collected in fluid reservoir 102 by clogging threaded tube 111. In other embodiments there is no mesh filter.

A plurality of different polymeric materials may be employed for constructing the drainage bag. Some examples of such polymeric materials include, without limitation, high density polyethylene, low density polyethylene polypropylene, medium density polyethylene or degradable olefinic materials.

As shown in FIGS. 2B, the flexible band 105 has two notches 116 cut into its end to form upper and lower prongs 115. Upper and lower prongs 115 have two protuberances 113 formed into them. In this embodiment disposable drainage bag 101 has sleeve 112 into which flexible band 105 with prongs 115 and their associated serrated distal protuberances 113 can be inserted. It is understood that the serrated protuberances are illustrative only. In other embodiments these protuberances could be circular, serrated or barbed, rectilinear buttons, hooks, pegs or other shapes. Sleeve 112 of disposable drainage bag 101 fits around flexible band 105 and couples the disposable drainage bag 101 to the flexible band 105. Flexible band 105 supports and holds open the disposable drainage bag 101 as shown in FIG. 2A.

Flexible band 105 is flexible in the horizontal direction and semi-rigid in the vertical direction. Since it is flexible in the horizontal direction flexible band 105 is able to flex in response to clinicians moving against or contacting the band allowing the clinician to approach surgical table 107 without blocking the disposable drainage bag system 110. In this embodiment the disposable support band 105 is made from plastic which has flexible properties although in other embodiments it can be made from other suitable materials. Disposable clamps 100A and 100B are substantially equivalent being mirror images of one another. As discussed in more detail below, disposable clamps 100A and 100B can be attached to the distal end of surgical table rail 106 of surgical table 107.

FIG. 2C shows detailed aspects of embodiments of the above flexible band 105 (herein referred to for ease of description of various components as flexible band 213), disposable drainage bag 101 (which is referred to in this example as disposable drainage bag 214), disposable clamp 200 (which can correspond to one of the disposable clamps 100A/100B) and accessory mount clamp 215. In the embodiment illustrated in FIG. 2C, the disposable clamp 200 has an upper ledge 205 with upper ledge chase 231, extension stabilizer 202. Further, the disposable clamp 200 has both a lateral surface (not shown) and a medial surface 209 forming rail acceptance channel 208.

In this embodiment, under surface 205a of upper ledge 205 together with upper ledge chase 231 and stabilizer extension 203 forms the upper surface of rail acceptance channel 208 while lower ledge 206 together with stabilizer extension 203 forming the lower surface of rail acceptance channel 208. In some embodiments, a lip 210 extends down from upper ledge 205 and forms one proximal surface of rail acceptance channel 208 while a lip 211 extends from lower ledge 206 and forms another proximal surface of rail acceptance channel 208. Distal surface 209 forms proximal surface of rail acceptance channel 208. In this embodiment threaded knob 201 engages with, and fits into, threaded hole (not shown) in lower ledge 206. In other embodiments the threaded hole can be formed in the upper ledge 205. A bolt or any other suitable fastener could also be used in place of the threaded knob 201, such as a cotter pin, threaded pin with lug, etc.

In the embodiment illustrated in FIG. 2C, when the surgical table rail (not shown) or mount arm 218 of accessory mount clamp 215 with fin 230 is engaged into the surgical rail acceptance channel 208 of disposable clamp 200 threaded knob 201 can be engaged into and through threaded hole (not shown) in lower ledge 206. The engagement of the threaded knob 201 into threaded hole (not shown) secures the disposable clamp to the surgical table rail (not shown) or accessory mount arm 218 via its fin 230. More specifically, it can be seen in this illustration that the fin 230 fits into upper ledge chase 231 when threaded knob is engaged into and through threaded hole in lower ledge 206 (not shown). After a surgical procedure is completed, threaded knob 201 can be disengaged from the hole in lower ledge 206 allowing disposable clamp 200 to disengage from mount arm 218 with fin 230 (or surgical table rail) and be removed.

In this embodiment when surgical table rail (not shown) or mount arm 218 with fin 230 is engaged into rail acceptance channel 208 of the disposable clamp with upper ledge chase 231 and stabilizer extensions 202 and 203, the disposable clamp 204 is protected from any torsion or rotational forces that may be encountered. In this embodiment disposable clamp 200 has flexible band acceptance slot 207 formed into one of its sides. Two through holes are formed into band acceptance slot 208 with one on the upper side of band acceptance slot 207 (visible) and one on its lower side (not visible in this illustration).

However, in other embodiments, more than two through holes may be formed into band acceptance slot 207 or only one through hole may be so formed in yet other embodiments. It should also be understood that although through holes are shown in this embodiment in other embodiments indentations can be formed in band acceptance slot 207. In this embodiment through hole 204 in band acceptance slot 207 is shown as being rectilinear but it is understood that through hole 204 can be of shapes differing from that shown in FIG. 2C. Alternatively, as noted above, in other embodiments rather than one or more through holes there can be indentations formed in band acceptance slot 207.

In this embodiment disposable flexible band 213 is fitted into sleeve 228 of disposable drainage bag 214 as shown in FIG. 2C. Flexible band 213 has notches 230 cut into its ends forming insertion prongs 229 with serrated protuberances 212. In this embodiment the maximum distance “Z” between serrated protuberances 212 is slightly larger than the height of band acceptance slot 207. As used herein, the phrase “slightly larger” is defined as being between 0.01 inches to 0.15 inches larger. The band 213 can also include one or more tabs that are configured to fit into slot(s) formed in a back side of the disposable clamp 200 to secure the to the band 213 to the disposable clamp 200. As the end of flexible band 203 is engaged into band acceptance slot 207 protuberances 212 are slightly depressed such that when protuberances 212 engage with through holes 204 the tension on them is released engaging protuberances 212 in slots 204 connecting flexible band 213 with disposable clamp 200. In other embodiments through holes 204 might be indentations sized to accept protuberances 212.

One embodiment of accessory mount clamp 228 as shown in FIG. 2C includes accessory mount clamp 215 comprising an upper arm 216 with hole 220, a lower arm 217 with threaded hole 221, semi-circular section 215 with mount arm 218 with fin 230 plus spacer 223 with through hole 224. Connecting rod 225 includes a threaded end 227 with a flat head on end 226. Connecting rod 225 can be pushed into and through hole 220 that is formed in the upper arm 216 and passed into hole 222 of spacer 223 and out through hole 224 and be engaged into threaded hole 221 in lower arm 217 thus affixing spacer 223 to accessory mount clamp 215. In this embodiment, accessory mount clamp 215 is formed as a monolithic structure out of plastic or metal although it is understood that each of the component parts could be assembled by gluing, press fitting or welding these parts together in any combination. Spacer 224 in this embodiment is shown as a rectilinear block although in other embodiments it could be square, circular, semi-circular or a “t” shaped monolith. The rail access channel 208 with extension stabilizers 202 and 203 of disposable clamp 200 can be engaged with mount arm 218 while mount arm fin 230 can be engaged into upper ledge chase 231 of accessory mount clamp 215.

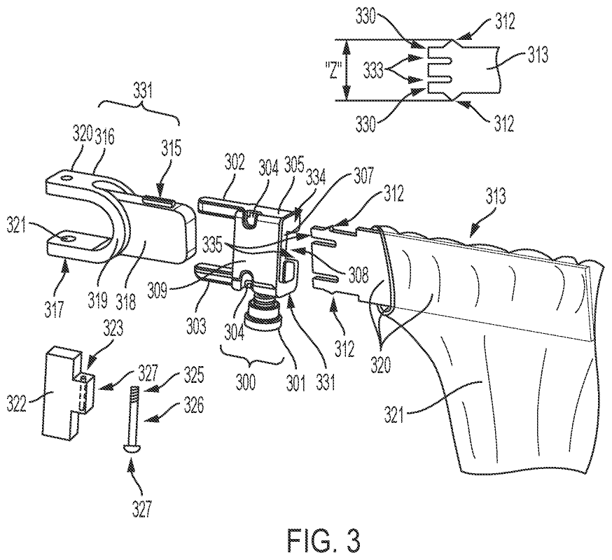

FIG. 3 shows another perspective view of an embodiment of a flexible band 313, disposable drainage bag 321, disposable clamp 300, accessory mount clamp 316, spacer 322 and rod 326. Flexible band 313 is engaged into sleeve 320 of drainage bag 321. In this embodiment disposable clamp 300 has upper ledge 305. The upper ledge 305, extension stabilizer 302 along with the proximal surface of clamp wall 309 form the distal surface of rail acceptance channel 308. In this embodiment, an upper ledge chase 334 is formed into an under surface of upper ledge 305.

Upper ledge 305 stabilizer extension 302 forms the upper surface of rail acceptance channel 308 while lower ledge 331 with stabilizer extension 303 forms the lower surface of rail acceptance channel 308. In some embodiments threaded knob 301, engages with, and fits into, a threaded hole (not shown) in lower ledge 331. When engaged into rail acceptance channel 308, threaded knob 301 engages accessory mount arm 318 via fin 315 within rail acceptance channel 308. In this embodiment flexible band 313 has two notches 333 cut into its end forming upper and lower prongs 300 with protuberances 312. The maximum distance between protuberances 312 of “Z” is slightly larger than the height of flexible band acceptance slot 307 of disposable clamp 300. As noted above, the phrase “slightly larger” as used herein is intended to indicate about 0.01 inches to about 0.15 inches larger. When the end of flexible band 313 is engaged into band acceptance slot 307 of disposable clamp 300, protuberances 312 are slightly depressed creating tension such that the engagement of protuberances 312 with holes 304 in the band acceptable slot 307 secures the disposable clamp 300 to flexible band 313.

FIG. 3 shows an embodiment of accessory mount clamp 331 comprising an upper arm 316 with hole 320, a lower arm 317 with threaded hole 321, a semi-circular section 319 with mount arm 318 with fin 315 plus spacer 322 with through hole 323. In this embodiment although spacer 322, with flange 324, is shown as a “t” shaped part it is understood that it could also be square, rectilinear, spherical or semi-spherical or other appropriate shapes. Connection rod 326 includes a threaded end 325 with a flat head on end 327. Connection rod 326 can be pushed into and through hole 320 in upper arm 316 and passed into through hole 323 in flange 324 of spacer 322 and out through the hole to be engaged into threaded hole 221 of lower arm 217, thereby engaging spacer 323 with accessory mount clamp 331.

In this embodiment the “u” shaped portion of accessory mount clamp 331 is formed as a monolithic structure out of plastic or metal or both although in some embodiments, the mount clamp 331 can be formed as a plurality of discrete component parts that are assembled together, e.g., via gluing, press fitting or welding. Rail access channel 308 with extension stabilizers 302 and 303 and upper ledge chase 334 of disposable clamp 300 can be engaged with mount arm 318 of accessory mount clamp 331.

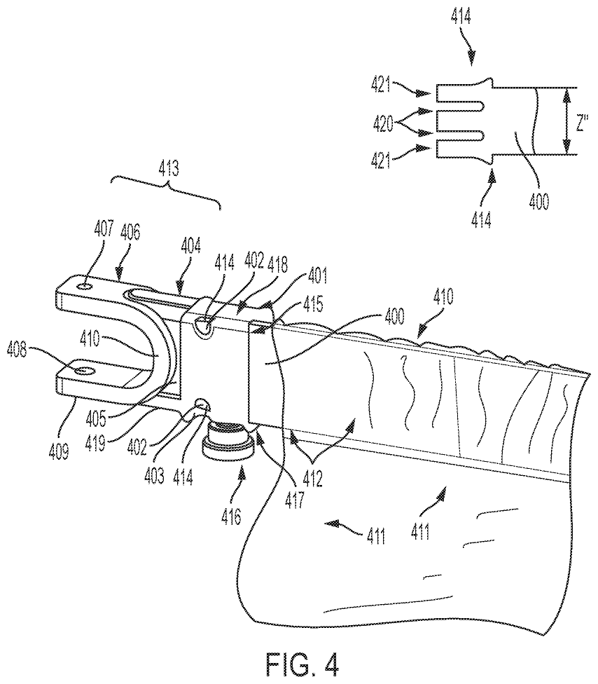

FIG. 4 illustrates an embodiment of a flexible band 400 fitted into sleeve 412 of a disposable drainage bag 411 engaged into disposable clamp 401, which in turn is connected with accessory mount clamp 413. In this embodiment, flexible band 400 is fully engaged into flexible band acceptance slot 415. Flexible band has notches 420 cut into its end to form upper and lower prongs 421. Protuberances 414 are formed on prongs 421. Distance “Z” between protuberances 414 is slightly larger than the height of band acceptance slot 415 of disposable clamp 418. As noted above, the phrase “slightly larger” as used herein indicates between about 0.10 and about 0.15 inches larger.

When the end of flexible band 400 is engaged into band acceptance slot 415, protuberances 414 are slightly depressed creating tension such that when protuberances 414 are engaged into through holes 414 flexible band 411 is thus secured to disposable clamp 418. An accessory mount clamp 413 includes an upper arm 406 with hole 407, a lower arm 409 with threaded hole 408 and a semicircular section 410 with extension arm 405. Mount arm 405 extends at right angles from semi-circular section 410. Rail acceptance channel of disposable clamp 401 formed by upper ledge 418 with stabilizer extension 404 and lower ledge 417 with stabilizer extension 419 can be engaged with mount arm 405 of accessory mount clamp 413. Threaded knob 416 can be engaged into threaded hole in lower ledge 417 of disposable clamp 410, thereby securing mount arm 405 to disposable clamp 401 which in turn secures accessory mount clamp 413 to disposable clamp 401.

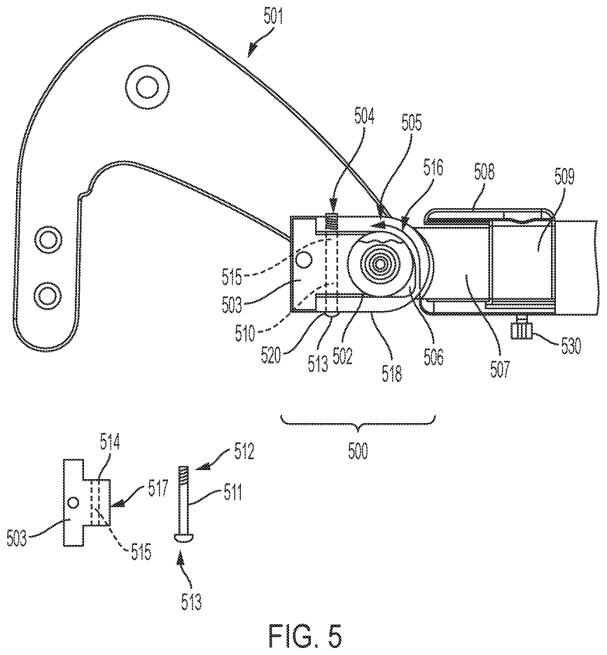

FIG. 5 illustrates an embodiment of the present teachings in which a mount 500 is attached to stirrup gas cylinder proximal mount lug 502 as well as to disposable clamp 509. Stirrup bracket 501, which mounts stirrup (not shown) to surgical table (not shown) has stirrup gas cylinder proximal mount lug 502 attached to it. Accessory mount clamp 516 includes an upper arm 505 with threaded hole 504, a semi-circular section 506 with mount arm 507 at right angles, and a lower arm 518 with hole 510. By passing rod 511 with threaded end 512 and flat head end 513 through hole 520 in lower arm 518 of accessory mount clamp 516 and into and through hole 514 of spacer 503 so that threaded end of rod 511 engages with threaded hole 504 of upper arm 505, accessory mount clamp 516 and spacer 503 can be connected around stirrup gas cylinder proximal mount lug 502.

With accessory mount clamp 516 connected to spacer 503, the surfaces of upper arm 505, semi-circular section 506, lower arm 518 and spacer 503 are snugged up against the surface of stirrup gas spring proximal mount lug 502. Disposable clamp 509 can be engaged with mount arm 507 securing it to stirrup gas cylinder mount lug 502.

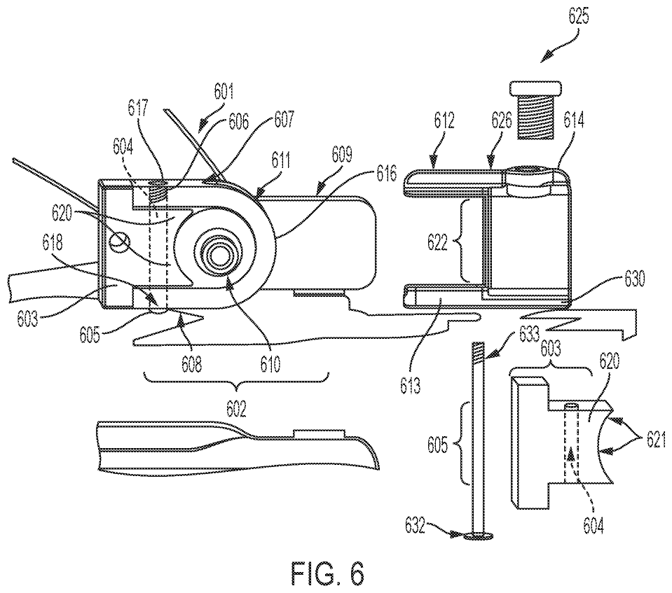

FIG. 6 illustrates an expanded view of an embodiment of a mount 602. The mount 602 includes a “t” shaped spacer 603 with flange 620a with semi-circular cut-out 621, attachment rod 605 with threaded end 633 and flat head end 632 and through hole 604 plus accessory mount clamp 611, which includes an upper arm 607 with threaded hole 617 and a lower arm 608 with hole 604. In this embodiment, spacer 603 flange 620 with semi-circular cutout is shaped so that its surface snugs up against the surface of mount lug 610, which is attached to stirrup bracket 601, which mounts stirrup (not shown) to surgical table (not shown).

In this embodiment, spacer 603 is shown as a “t” shaped monolith however, in other embodiments, spacer 603 could be square or rectilinear while flange 620 could be rectilinear, round or obloid in shape. Accessory mount clamp 611 includes a top arm 607 with a threaded hole 617, a semi-circular section 616 and a lower arm 608 with hole 618. In this embodiment accessory mount clamp 611 is shown as a monolith but it could be made of a number of discrete component parts that could be pressed fit, welded or glued together. Spacer 603 could be made of plastic or metal or combination of these materials, such as those listed above in connection with the previous embodiments.

Spacer 603 has through hole 604 in flange 620 with semi-circular cut out 621 allowing the surface of the flange 620 to mate closely and intimately with the proximal surface of gas cylinder proximal mount lug 602. Accessory mount clamp 611 also includes a mount arm 609. Disposable clamp 630 has rail acceptance channel 622 formed by upper and lower stabilizer extensions 612 and 613 and the lower surface of upper ledge 626 and upper surface of lower ledge 630, respectively. In this embodiment, rail acceptance channel 622 of disposable clamp 630 can be engaged onto mount arm 609 of accessory mount clamp 611. Threaded hole 614 is formed in top ledge of 626 into which threaded knob 625 can be engaged. Mount arm 609 of accessory mount 602 can be engaged into rail clamp acceptance channel 622 of disposable clamp 630. When threaded knob 625 is engaged in threaded hole 614, disposable clamp 630 is secured to the mount arm of reusable mount 602. In this embodiment, threaded hole 614 is formed in top ledge 626, but in other embodiments, it could be formed in a bottom ledge of disposable clamp 630.

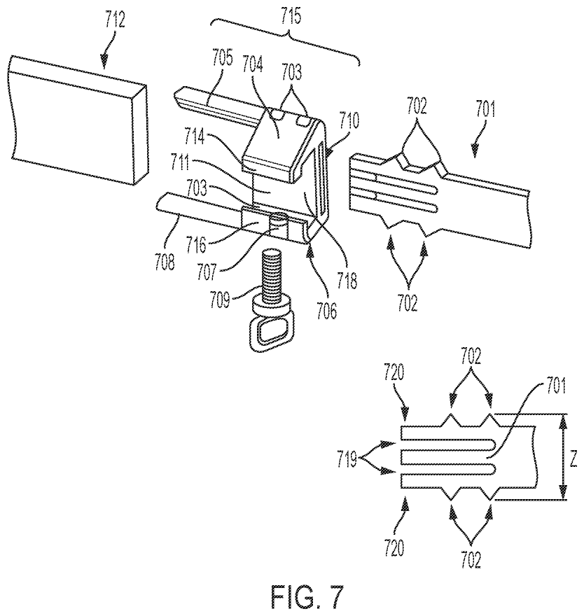

FIG. 7 provides details of connections between surgical table side rail 712, disposable clamp 715 and flexible band 701. Disposable clamp 715 has upper ledge 704 with lip 714, lower ledge 706 with lip 716, proximal clamp wall 718, an upper stabilizer extension 705, a lower stabilizer extension 708 and band acceptance slot 710 with through holes 703. This embodiment shows four through holes but other embodiments could have at least two through holes. Yet other embodiments could have at least two indentations instead of through holes. Lower ledge 706 has threaded hole 707 into which threaded knob 709 can be engaged. Rail access channel 711 of disposable clamp 715 is formed from proximal clamp wall 718, lower surface of upper ledge 704, upper surface of lower ledge 706, upper lip 714, lower lip 716, upper extension 705, and lower extension 708.

Surgical table rail 712 can be engaged into rail acceptance channel 711 and threaded knob 709 can be engaged into and through threaded hole 707 securing disposable clamp 715 to surgical table rail 712. Flexible band 701 has notches 719 formed or cut into its end to form prongs 720. Prongs 720 have protuberances 702. This embodiment shows two protuberances formed on each of prongs 720 but other embodiments may have one protuberance on each prong while yet other embodiments may have three or more protuberances. Distance “z” between prongs 720 is slightly larger than the height of band acceptance slot 710. The phrase “slightly larger” is defined as between 0.10 to 0.15 inches larger. When the end of flexible band 701 is engaged into band acceptance slot 710, protuberances 702 are slightly depressed. When protuberances 702 are engaged into holes 703 of disposable clamp 715, flexible band 701 is secured to disposable clamp 715.

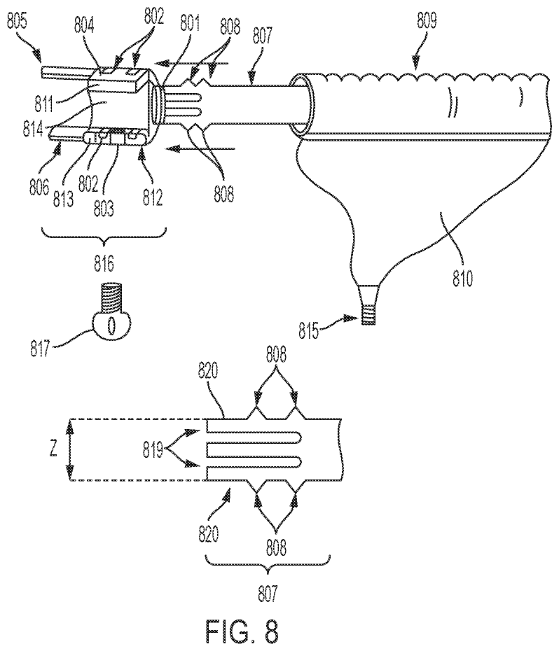

FIG. 8 provides details of flexible band 807, disposable drainage bag 810 with sleeve 809 and disposable clamp 816. Disposable clamp 816 has an upper ledge 804 with lip 811, a lower ledge 812 with lip 813, a proximal clamp wall 814, an upper stabilizer extension 805, a lower stabilizer extension 806, and a band acceptance slot 801 with through holes 802. This embodiment shows four through holes but other embodiments could have at least two through holes. Other embodiments could have at least two indentations instead of through holes. The lower ledge 812 has threaded hole 803 into which threaded knob 817 can be engaged. The end of flexible band 807 has two notches 819 cut into it forming prongs 820 with protuberances 808. This embodiment shows two protuberances 808 formed into each prong 820 but in other embodiments prong 820 may have one or more protuberances 808. The distance “z” is slightly larger than the height of band acceptance slot 801. As noted above, the phrase “slightly larger” is intended to indicate that the distance “z” is larger than the height of band acceptance slot 801 by an amount in a range of about 0.10 to 0.15 inches.

When the end of the flexible band 820 is engaged into band acceptance slot 801 protuberances 808 are slightly depressed such that when protuberances 808 are engaged into holes 802 of disposable clamp 816 flexible band 829 is secured to disposable clamp 816. Disposable drainage bag 810 has sleeve 809 which can be fitted onto flexible band 807. Disposable drainage bag 810 and sleeve 809 can be made of clear plastic. Threaded tube 815 is attached to the bottom of drainage bag 810 to allow fluid to flow into fluid collection reservoir (not shown).

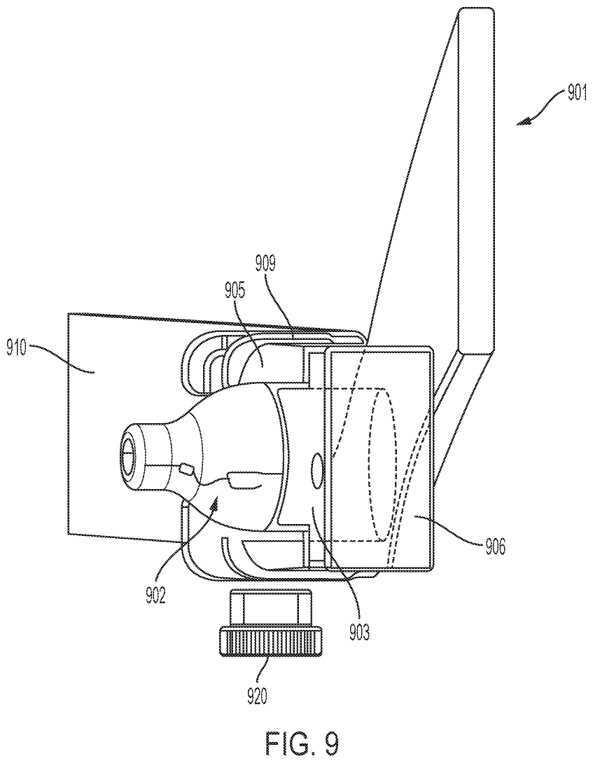

FIG. 9 illustrates some aspects of an embodiment of an accessory mount mechanism attached to a protrusion extending from surgical equipment in particular a lithotomy stirrup. This includes the distal surface of flange 903 of spacer 906 which fits snuggly against the proximal surface of stirrup gas cylinder mount lug 902, which is attached to stirrup mount bracket 901. The proximal surface of accessory mount clamp 905 fits snuggly against the upper, lower and distal surface of stirrup cylinder gas lug 902. Flexible band 910 is attached to disposable clamp 909 which in turn is attached to the accessory mount mechanism mount arm (not shown).

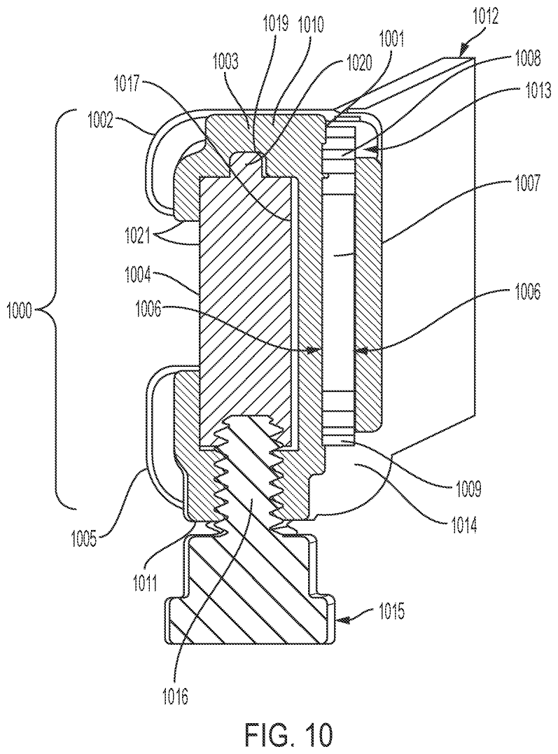

FIG. 10 shows a cut-away view of the disposable clamp 1000 with upper ledge 1010 and lower ledge 1011, lower lip 1005 and upper lip 1002, clamp wall 1001 with inner wall 1017, band acceptance slot 1006 with flexible band 1012 engaged in said slot with lower prong protuberance 1009 and upper prong protuberance 1013 of flexible band 1012 engaged in through holes 1014 and 1013. Rail acceptance channel 1021 is formed by an upper ledge 1010, a lower ledge 1011, a lower lip 1005, an upper lip 1002 and an inner wall 1017 of disposable clamp 1000. Upper ledge chase 1019 is cut or formed into the lower surface of upper ledge 1010. Accessory mount arm 1004 is shown engaged in rail acceptance channel while fin 1020 is engaged into upper ledge chase 1019. Threaded knob 1015 is engaged into and through threaded hole 1016 of lower ledge 1011 securing mount arm 1004 to disposable clamp 1000.

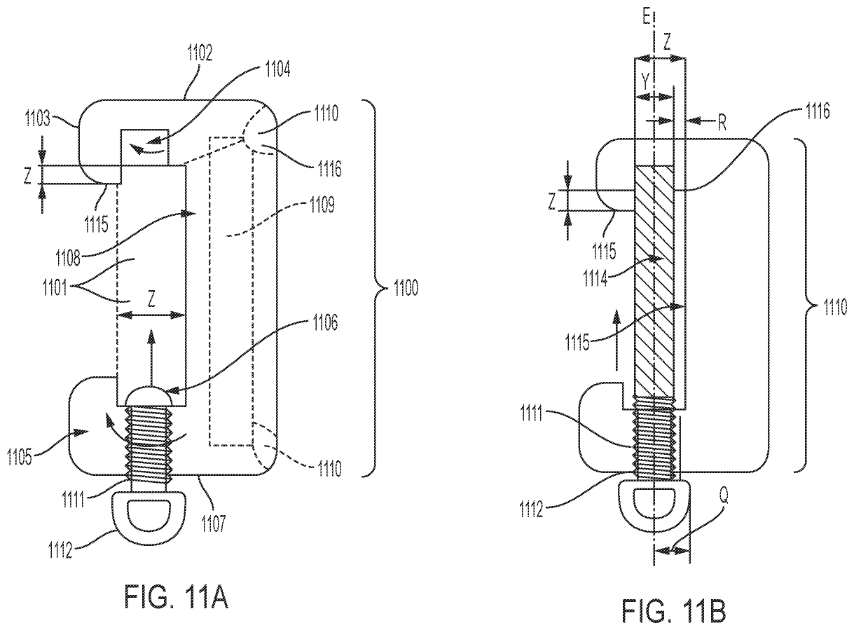

FIGS. 11A and 11B illustrate one cutaway view of one embodiment of the disposable clamp 1100 with upper ledge 1102, lower ledge 1107, upper lip protrusion 1103 with engagement surface 1115, lower lip protrusion 1105, upper ledge chase 1104, proximal clamp wall 1115 with notch 1116 making up rail acceptance channel 1101. In one embodiment, rail acceptance channel 1101 has width “Z”, e.g., about 0.4 inches. In some embodiments the upper surface of notch 1116 is at a distance “N” from lower edge of engagement surface 1115, where in some embodiments N may be equal to about 0.125 inches. Disposable clamp 1100 has band acceptance slot 1109 with slot through holes 1110, and threaded hole 1111 in lower ledge 1107 with threaded knob 1112. In this embodiment center line “E” of threaded hole 1111 in lower ledge 1107 is offset from center line “E” of disposable clamp 1110 by a distance “Q.” Width of rail 1114 less than the width of rail acceptance channel 1101 by an amount “R,” where in some embodiments “R” may be equal to about 0.15 inches and width “Y” of chase 1114 formed into lower surface of upper ledge 1102 may be equal to about 0.25 inches. When fully engaged into threaded hole 1111 threaded knob 1112 pushes up on table rail 1114 with width “Y” into chase 1104.

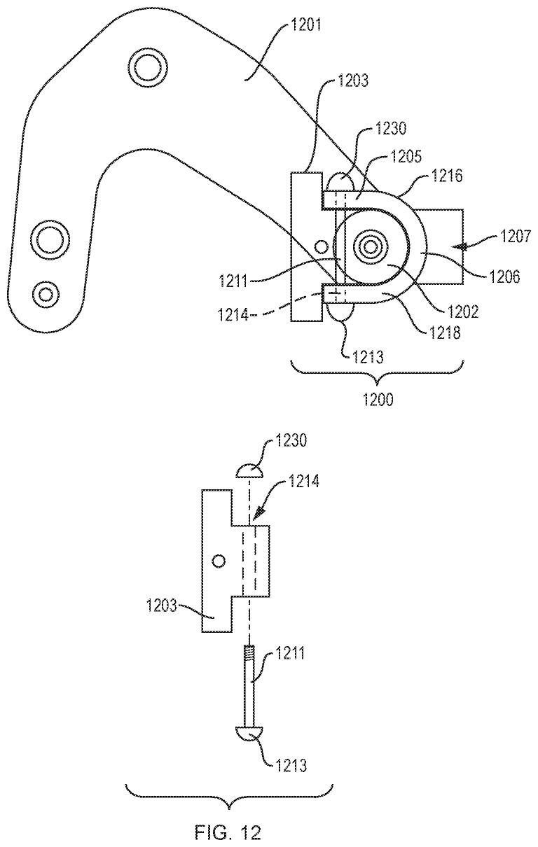

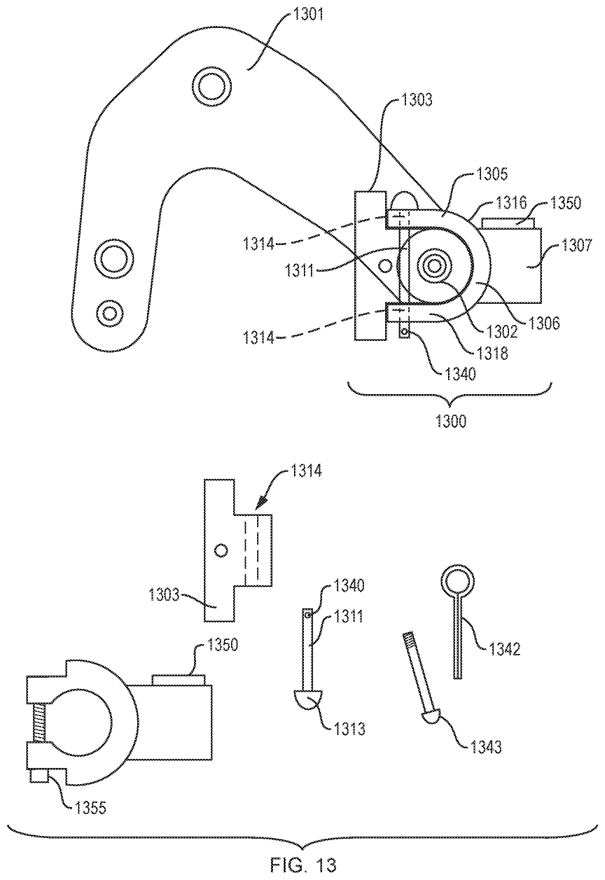

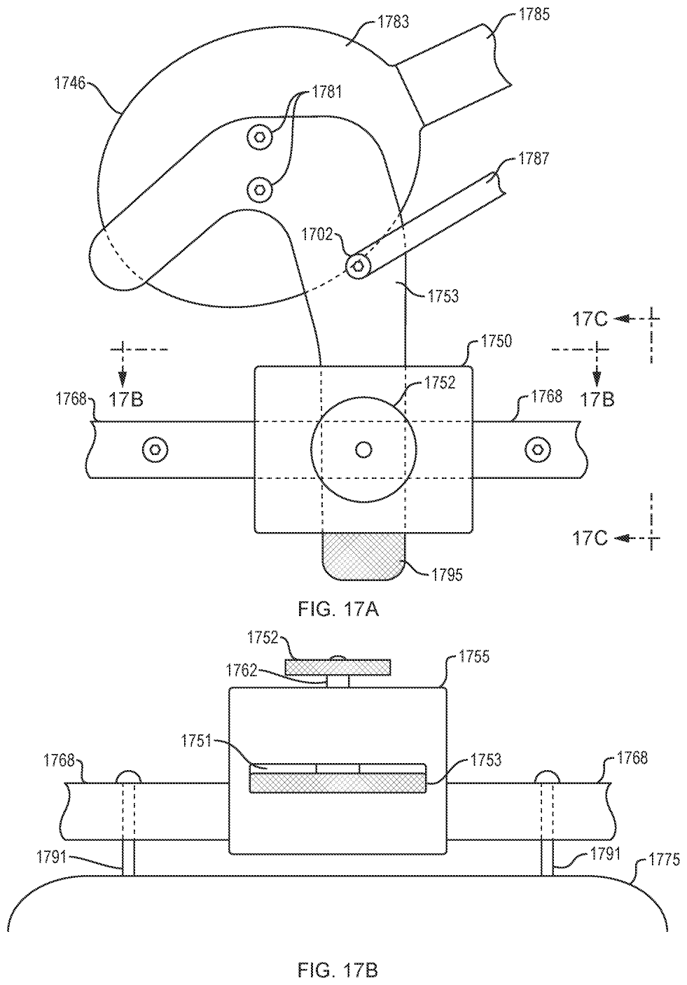

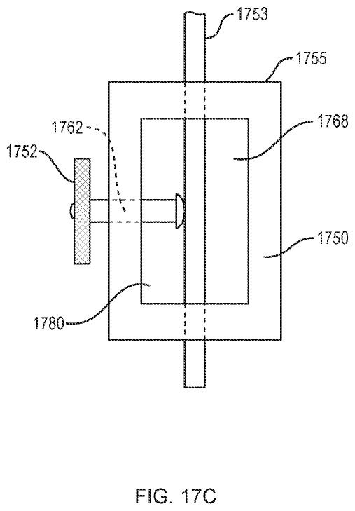

FIGS. 12 and 13 show embodiments that allow additional instruments to be secured to surgical table at positions other than the rails of the surgical table. FIG. 12 illustrates an embodiment of the present teachings in which stirrup bracket 1201 is attached to stirrup gas cylinder proximal mount lug 1202 of a stirrup mount 1200. Stirrup bracket 1201, which mounts stirrup (not shown) to surgical table (not shown), for example to a rail of a surgical table, has stirrup gas cylinder proximal mount lug 1202 attached to it. The mount bracket 1201 of mount 1200 can be attached to an accessory clamp 1216. See FIG. 17 for details of mounting such a bladed accessory to a surgical table. Accessory mount clamp 1216 includes an upper arm 1205 with hole 1214, a semi-circular section 1206 with mount arm 1207 at right angles and a lower arm 1218 with hole 1214. By passing rod 1211 with flat head end 1213 through hole 1214 in lower arm 1218 of accessory mount clamp 1216 and into and through hole 1214 so that end of rod 1211 passes through hole 1214 of upper arm 1205, cap 1230 can then be permanently attached to upper end of rod 1211 thereby securing the rod 1211 to the accessory mount clamp 1216 with spacer 1203 connected around stirrup gas cylinder proximal mount lug 1202. In another embodiment, a rod 1211, or other suitable fastener, can be used to secure the upper arm 1205 to the lower arm 1218 without using a spacer. Examples of suitable fasteners include a bolt, other suitable fasteners could include but are not limited to a threaded screw or a bolt with a cotter pin. Accessory clamp (not shown) can be attached to disposable clamp/mount arm 1207 while instruments or accessories (not shown) can be secured to the accessory clamp. Examples of instruments and accessories include a drainage bag 101, instruments including scopes, retractors, forceps, arm holders and anesthesia screens.

FIG. 13 illustrates yet another embodiment of the present teachings in which a mount 1300 is attached to stirrup gas cylinder proximal mount lug 1302. Stirrup bracket 1301, which mounts stirrup (not shown) to surgical table (not shown) has stirrup gas cylinder proximal mount lug 1302 attached to it. Accessory mount clamp 1316 includes an upper arm 1305 with hole 1314, a semi-circular section 1306 with mount arm 1307 at right angles and a lower arm 1318 with hole 1314. By passing rod 1311 with flat head end 1313 through hole 1314 in lower arm 1318 of accessory mount clamp 1316 and into through hole 1314 so that end of rod 1311 with pin hole 1340 passes through hole 1314 of upper arm 1305, placing cotter key 1342 in pin hole 1340 and placing securement pin 1343 in the hole in cotter key 1342, the accessory mount clamp 1316 becomes secured to spacer 1303 connected around stirrup gas cylinder proximal mount lug 1302. Accessory clamp (not shown) can be attached to mount arm 1307 while instruments (not shown) can be secured in accessory clamp (not shown). A fin 1350 can be included at top portion of the accessory mount clamp 1316. To provide a more secure connection. In another embodiment, a rod 1355, or other suitable fastener, can be used to secure an upper portion of the mount 1300 to a lower portion of the mount 1300 without using a spacer, as shown in the lower left portion of FIG. 13.

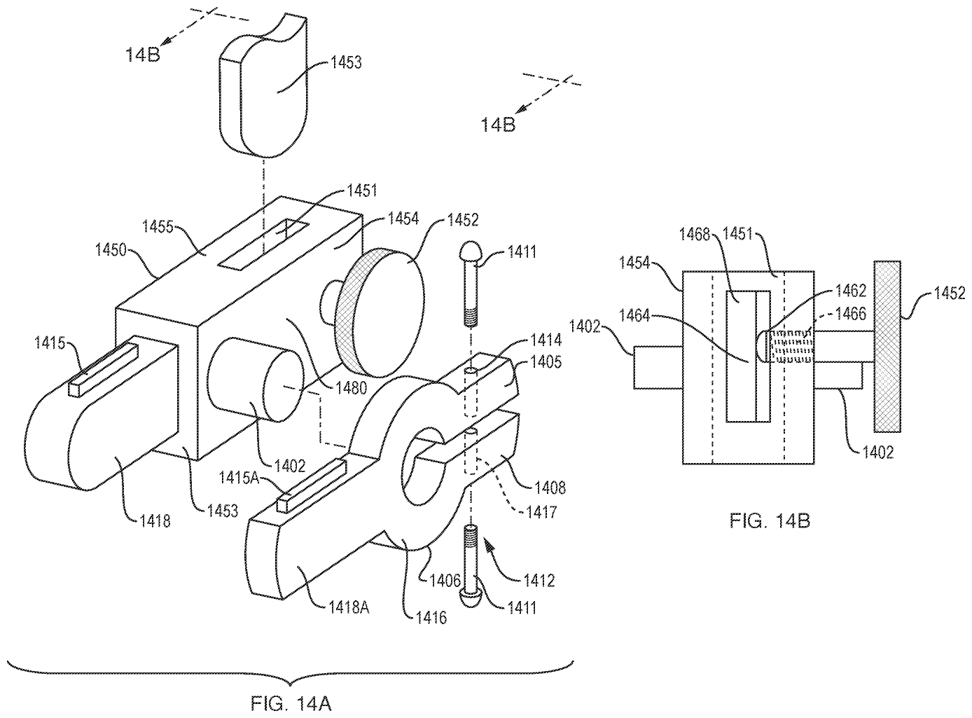

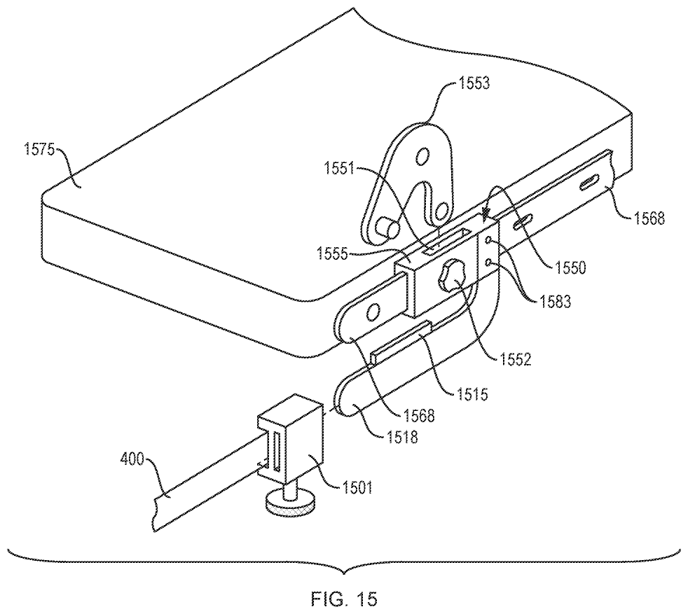

FIG. 14 illustrates an embodiment of surgical table accessory rail clamp 1450 having accessory mount arm 1418 formed into distal surface 1453 with fin 1415, to which an accessory can be mounted. In this embodiment, surgical table rail accessory clamp 1450, accessory mount arm 1418 with fin 1415 can be formed as a monolithic structure out of plastic or metal although it is understood that each of the component parts could be assembled by gluing, press fitting or welding these parts together in any combination. In other embodiments a mount lug 1402 can be formed in lateral surface of 1454 of surgical table accessory rail clamp 1450 allowing accessory mount clamp 1416 to removably attach to the clamp 1450. Mount arm 1418A with fin 1415A is formed into accessory mount clamp 1416 with circular section 1406. Hole 1415 with threaded end 1414 is formed through lower arm 1408 and upper arm 1405 of mount accessory mount clamp 1416. Bolt 1411 with threaded end 1412 can be engaged in hole 1415 with threaded end 1414 of upper arm 1405, thereby securing accessory mount clamp 1416 to mount lug 1402 of surgical table accessory rail clamp 1450. Other embodiments of bolt 1411 could include a cotter pin method of securing bolt 1411 and thus affixing accessory mount clamp 1416 to mount lug 1402. The rail access channel of disposable clamp with stabilizers (not shown) can be engaged and secured to accessory mount arm 1418A with fin 1415A. In this embodiment, rail clamp 1450 with tightening knob 1452 has accessory blade acceptance slot 1451 formed into its upper surface 1455 to accept surgical accessory mount blade 1453. The acceptance slot 1451 can be any type of opening, such as a round opening, oval opening, rectilinear opening, etc. Knob 1452 has a threaded stud 1466 which can be screwed into and though a hole (not shown) in a front surface 1480 of the accessory clamp 1450, thereby securing the accessory blade 1453 to the rail clamp 1450. Further tightening knob 1452 in turn pushes against surgical table side rail 1468 securing said clamp to the surgical table (not shown). Channel 1462 can be formed into proximal side surface of the knob 1452 and can be sized to accept surgical table side rail 1464. In other embodiments rail clamp 1450 may have accessory round post acceptance hole formed into its upper surface 1455 to accept accessory mount post (not shown). In yet other embodiments, rail clamp 1450 may have both a blade acceptance slot and accessory round post acceptance hole formed into upper surface 1455.

FIG. 15. illustrates yet another aspect of the present disclosure. Surgical table 1575 has surgical table side rail 1568 attached to the surgical table by standoffs (not shown). Accessory mount blade 1553 may be inserted into accessory slot 1551 formed in an upper surface 1555 of surgical table accessory rail clamp 1550 and secured to the clamp by means of tightening knob 1552 that can have a threaded screw (not shown) attached to it. The surgical table accessory rail clamp 1550 comprises accessory mount arm 1518 formed or attached into its distal end thereof, as illustrated in FIG. 15. The accessory mount arm 1518 can comprise a fin 1515. Accessory mount arm 1518 and fin 1515 can be formed as a monolithic structure out of plastic or metal although it is understood that each of the component parts could be assembled by gluing, press fitting or welding these parts together in any combination and is attached to distal end of surgical table rail accessory clamp 1550. The rail access channel of disposable clamp 1516 with stabilizers can be engaged and secured to accessory mount arm 1518 with fin 1515. In this embodiment, surgical table accessory rail clamp 1550 comprises a tightening knob 1552. In other embodiments rail clamp 1550 may have an accessory round post acceptance hole formed into its upper surface 1555 to accept accessory mount post (not shown). In yet other embodiments rail clamp 1550 may have both a blade acceptance slot and accessory round post acceptance hole formed into upper surface 1555. Bolts 1583 can be used to secure mount arm 1518 with fin 1515 to accessory clamp 1550. A disposable clamp 1501 can be removably secured to mount arm 1518 of accessory clamp 1550.

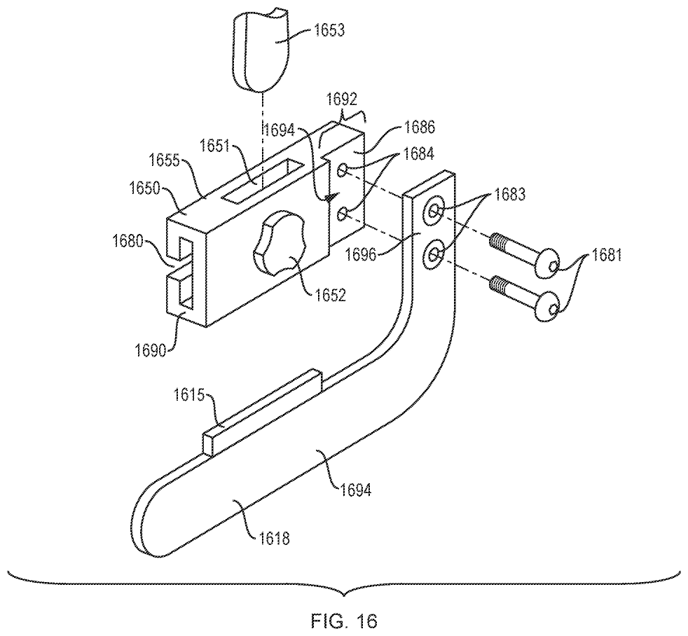

FIG. 16 illustrates one embodiment of surgical table accessory rail clamp 1650 comprising rail acceptance channel 1680, knob 1652, and accessory blade acceptance slot 1651 formed into and through a top surface 1655 that can accept surgical accessory blade mount 1653, and threaded holes 1684. Channel 1680 is formed into and through the distal surface 1690 of accessory clamp 1650. Mount arm 1618 with fin 1615 can be affixed to surgical table accessory rail clamp 1650 by passing threaded bolts 1681 through holes 1683 in mount arm 1618 and into threaded holes 1684 formed in the clamp 1650. Knob 1652 having a threaded stud (not shown) attached thereto can be screwed into a hole (not shown) formed into the front surface of accessory clamp 1650. Cutaway 1692 can be formed into the proximal end of accessory clamp 1650. Holes 1684 can be formed into the front surface 1694 of cutaway 1692. Mount arm 1618 has horizontal portion 1694 and vertical portion 1696. Holes 1683 can be formed into vertical portion 1696 of mount arm 1618. Threaded bolts 1681 can be fitted into holes 1683 securing mount arm 1618 to accessory clamp 1650.

In other embodiments surgical table rail clamp 1650 may have an accessory round post acceptance hole formed into its upper surface 1655 to accept accessory mount post (not shown). In yet other embodiments rail clamp 1650 may have both a blade acceptance slot and accessory round post acceptance hole formed into upper surface 1655 of surgical table accessory rail clamp 1650.

FIGS. 17A-17C show a bladed leg holding stirrup accessory mounted via accessory clamp 1750 to surgical table side rail 1768 of surgical table 1755. A portion of stirrup 1746 is shown with actuation joint 1783 that permits distal actuation and multiplane motion of the stirrup using the action of a distal handle (not shown) at the distal end (not shown) of the stirrup. Support tube 1785, upon which a leg holding boot (not shown) may be attached, is connected to actuation joint 1783. Actuation joint 1783 can be attached to blade mount 1753 by bolts 1781. Stirrup gas cylinder 1787 can be attached to a mount blade 1753 via mount lug 1702 formed into or attached to the mount blade 1753. Accessory clamp 1750 has channel 1780 (see FIG. 17C) which is sized to accept surgical table rail 1768. Surgical table rail 1768 can be attached to surgical table 1775 by standoffs 1791. Accessory clamp 1750 has acceptance slot 1751 formed into and through top surface 1755 (see FIG. 17B). Mount blade 1753 has lower mount tang 1795 which can be fitted into and through acceptance slot 1751 of accessory clamp 1750. Threaded screw 1762 can be attached to knob 1752 and can be fitted and screwed into a through hole (not shown) formed into and through the front surface of accessory clamp 1750. By screwing threaded screw 1762 into and through a hole (not shown) in a front surface of accessory clamp 1750, lower mount tang 1795 of mount blade 1753 can secured to the accessory clamp 1750, which in turn secures accessory clamp 1750 to surgical table side rail 1768 and thus to surgical table 1775.

Although certain illustrative embodiments are described in detail above; many embodiments, variations and modifications are possible that would be within the scope and spirit of this disclosure as described herein and as defined the claims that follow.

What is claimed is:

1. A mount configured to attach to a mount lug, the mount comprising:
accessory mount clamp comprising a mount arm extending from the accessory mount clamp; and
a spacer comprising a hole extending through the spacer through which a rod can be passed to secure the spacer to the accessory mount clamp, thereby connecting the accessory mount clamp to the mount lug.

2. A mount configured to attach to a mount lug, the mount comprising:
accessory mount clamp comprising mount arm extending from the accessory mount clamp; and
a fastener to connect the accessory mount clamp to the mount lug.

3. The mount of claim 1, wherein the accessory mount clamp comprises an upper arm having a hole formed therein, semicircular section, and a lower arm having a hole formed therein.

4. The mount of claim 1, wherein the proximal mount lug is disposed on a stirrup bracket.

5. The mount of claim 1, wherein the spacer includes a semi-circular cut-out that is configured to mate with a proximal surface of the mount lug.

6. The mount of claim 1, wherein the mount arm is configured to attach to a disposable clamp to which an accessory can be mounted.

7. The mount of claim 5, wherein the disposable clamp includes a rail acceptance channel that is configured to engage with the mount arm.

8. The mount of claim 1, further comprising a cap that attaches to the upper end of rod to secure the rod to accessory mount clamp.

9. The mount of claim 1, further comprising a cotter key that is configured to extend through hole formed in the rod to secure the accessory mount clamp to the spacer, thereby securing the accessory mount clamp to the proximal mount lug.

10. The mount of claim 9, further comprising a securement pin that secures the cotter key to the rod.

11. The mount of claim 1, wherein the accessory mount clamp is a monolith.

12. The mount of claim 1, wherein the accessory mount clamp is made of discrete parts.

13. A mechanism for attaching a first accessory to a second accessory attached to a surgical table comprising:
an accessory mount clamp configured to engage with a proximal mount lug, the accessory mount clamp having a hole formed therein;
a mount arm disposed adjacent to the accessory mount clamp;
a spacer connectable to the accessory mount clamp; and
a rod configured to pass through holes formed in an upper portion and lower portion of the accessory mount clamp and configured to pass through holes formed in the spacer thereby connecting the spacer and the accessory mount clamp around the proximal mount lug.

14. The mount of claim 13, wherein the second accessory is a leg holder accessory.

15. The mount of claim 13, wherein the first accessory is a surgical bag.

16. The mount of claim 13, wherein the mount arm is configured to attach to a disposable clamp to which the accessory can be mounted.

17. The mount of claim 16, wherein the disposable clamp includes a rail acceptance channel that is configured to engage with the mount arm.

18. A mechanism for attaching a first accessory to a second accessory attached to a surgical table comprising:
an accessory mount clamp configured to engage with a proximal mount lug, the accessory mount clamp having a hole formed therein;
a mount arm disposed adjacent to the accessory mount clamp; and
a fastener to connect the accessory mount clamp to the mount lug.

19. The mount of claim 18, wherein the second accessory is a leg holder accessory.

20. The mount of claim 18, wherein the first accessory is a surgical bag.

21. The mount of claim 18, wherein the mount arm is configured to attach to a disposable clamp to which the accessory can be mounted.

22. The mount of claim 21, wherein the disposable clamp includes a rail acceptance channel that is configured to engage with the mount arm.

23. An accessory clamp configured to be secured to a surgical table rail of a surgical table comprising:
an accessory slot configured to receive a mounting member;
a channel formed therein configured for attaching to the surgical table rail; and
an accessory mount arm formed into distal surface of the accessory clamp to which an accessory can be mounted.

24. The accessory clamp of claim 23, wherein the extended portion comprises a fin.

25. The accessory clamp of claim 34, wherein the accessory mount clamp comprises an upper arm, lower arm, circular portion and rod configured to secure the adapter to the mount lug.

26. An accessory clamp configured to be secured to a surgical table rail of a surgical table comprising:
an accessory slot configured to receive a mounting member;
a channel formed in the accessory clamp configured for attaching to the surgical table rail; and
a mount arm attached to the accessory clamp and configured to receive a disposable clamp mounted thereto.

27. The accessory clamp of claim 26, wherein the mount arm comprises an extended portion.

28. The accessory clamp of claim 27, wherein the extended portion comprises a fin.

29. The accessory clamp of claim 26, wherein the mount arm comprises a cutaway portion for receiving an upper portion of the mount arm.

30. The accessory clamp of claim 26, wherein the mount arm comprises holes formed therein for receiving bolts that are configured to secure the mount arm to the accessory clamp.

31. The accessory clamp of claim 26, further comprising a knob configured to secure the mounting member to the accessory clamp.

32. The accessory clamp of claim 13, wherein the accessory mount clamp extends at least partially around the proximal mount lug.

33. The accessory clamp of claim 18, wherein the accessory mount clamp extends at least partially around the proximal mount lug.

34. The accessory clamp of claim 23, further comprising a lug and an accessory mount clamp configured to attach to the lug, wherein the accessory mount adapter comprises an extended portion configured to accept an accessory.

35. The accessory clamp of claim 23, wherein the mounting member is an accessory blade.

36. The accessory clamp of claim 26, wherein the mounting member is an accessory blade.
